(19) United States
(12) Patent Application Publication
(10) Pub. No.: US 2025/0319096 A1
(43) Pub. Date: Oct. 16, 2025

(54) METHODS AND COMPOUNDS FOR TREATING INFLAMMATION

(71) Applicant: PANTHERICS INCORPERATED, Stoughton, WI (US)

(72) Inventors: Douglas C. Stafford, Stoughton, WI (US); Alexander E. Arnold, Mikwaukee, WI (US)

(21) Appl. No.: 18/698,676

(22) PCT Filed: Sep. 22, 2022
(86) PCT No.: PCT/US2022/044401

§ 371 Date: Apr. 4, 2024

Related U.S. Application Data
(60) Provisional application No. 63/253,208, filed on Oct. 7, 2021.

(51) Int. Cl. A61K 31/5517; A61P 11/06; C07D 487/04

(52) U.S. Cl. CPC A61K 31/5517; A61P 11/06; C07D 487/04

(57) ABSTRACT

Methods of reducing inflammation in a subject in need thereof are described, such as a subject having an autoimmune disease, by administering an effective amount of a compound or a pharmaceutically acceptable salt thereof.

### CROSS-REFERENCE TO RELATED APPLICATIONS

The present application claims the benefit of U.S. Provisional Patent Application No. 63/253,208, filed on Oct. 7, 2021, which is incorporated herein by reference.

### FIELD OF THE INVENTION

The present invention relates to compounds and methods for treating inflammation, and in particular, where a higher local concentration of compound is achieved as compared to the concentration in the blood, regardless of the route of administration

### BACKGROUND

Around 1.6 million residents in the USA are estimated to have an Inflammatory Bowel Disease (IBD) as reported in 2014 (The Facts About Inflammatory Bowel Disease, Crohn's & Colitis Foundation of America, 2014, worldwideweb.ccfa.org). 2.5 million people in Europe are estimated to have IBD. With the corresponding lifelong need for medical care there is a substantial cost for current IBD patient health care. Further, the majority of IBD patients are diagnosed early in life and the incidence continues to rise; therefore, the effect of IBD on healthcare systems will rise exponentially. Moreover, IBD has emerged in newly industrialized countries in Asia, South America and Middle East and has evolved into a global disease with rising prevalence in every continent, Kaplan, “The global burden of IBD: from 2015 to 2025.” Nature Reviews Gastroenterology & Hepatology 12:720-727, 2015 [1].

New and effective ways are needed to treat IBD and other inflammatory diseases.

### SUMMARY OF THE INVENTION

In one embodiment, the present invention provides a method of reducing inflammation in a subject in need thereof, including but not limited to a subject having an autoimmune disease, by administering an effective amount of a compound (e.g. compound PI320, shown below), or a pharmaceutically acceptable salt thereof. In some embodiments, the autoimmune disease is selected from an Inflammatory Bowel Disease, an inflammatory lung disease, and an inflammatory skin disease. In one embodiment, the compound may be part of an oral or an aerosol formulation. In one embodiment, the compound may be part of a topical formulation. In a preferred embodiment, a higher local concentration of compound is achieved as compared to the concentration in the blood, regardless of the route of administration.

In one embodiment, the present invention contemplates a compound PI320 having the structure:

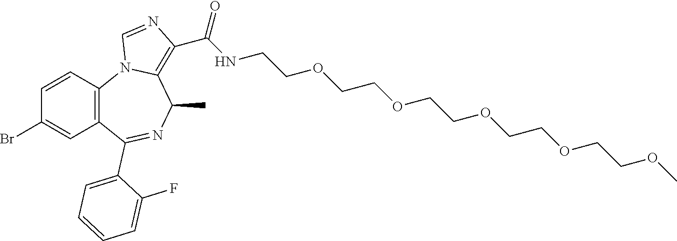 [SMILES: COCCOCCOCCOCCOCCNC(=O)c1ncn2c1[C@@H](C)N=C(c1ccccc1F)c1cc(Br)ccc1-2]

or a pharmaceutically acceptable salt thereof. In one embodiment, the present invention also contemplates a method of reducing inflammation comprising administering an effective amount of the compound (shown above), or a pharmaceutically acceptable salt thereof, to a subject in need thereof (including but not limited to human subjects). In one embodiment, said subject has inflammation of the gastrointestinal tract. In one embodiment, said administering is oral (e.g. for treatment of inflammation in the gut). In one embodiment, said administering is by aerosol (e.g. for treatment of inflammation in the airways, including the lungs). In one embodiment, said administering is topical (e.g. for treatment of the skin). In one embodiment, subject has inflammation due to an autoimmune disease. In one embodiment, autoimmune disease is inflammatory bowel disease. In one embodiment, said inflammatory bowel disease is selected from the group consisting of Crohn's disease and ulcerative colitis. In one embodiment, said subject has an inflammatory skin disorder. In one embodiment, said inflammatory skin disorder is atopic dermatitis. In one embodiment, said subject has an inflammatory lung disease. In one embodiment, said inflammatory lung disease is asthma. In a preferred embodiment, a higher local concentration of compound is achieved as compared to the concentration in the blood, regardless of the route of administration.

In one embodiment, the present invention contemplates a method of treating overactive bladder syndrome comprising administering an effective amount of the compound (shown above), or a pharmaceutically acceptable salt thereof, to a subject in need thereof.

In yet another embodiment, the present invention contemplates a compound having the structure:

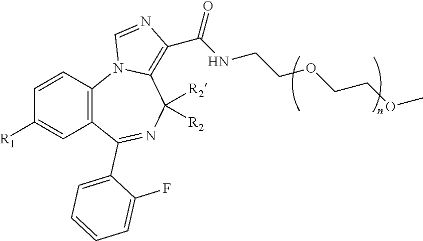

or a pharmaceutically acceptable salt thereof, wherein R₁ is H, Cl, Br, —OCH₃, —C≡CH, or cyclopropyl; R₂ and R₂′ are each independently H, D, C₁₋₄alkyl, cyclopropyl; or R₂ and R₂′, together form a substituted or unsubstituted ring, and n is any number between 1 and 15. In one embodiment, the compound has the structure:

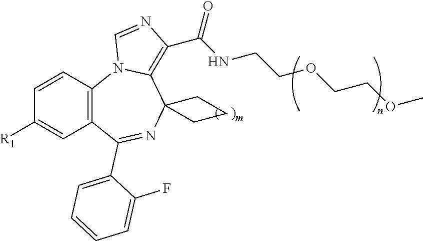

wherein m is any number between 0 and 3. In one embodiment, the present invention also contemplates a method of reducing inflammation comprising administering an effective amount of the compound (e.g. any of the compounds shown above), or a pharmaceutically acceptable salt thereof, to a subject in need thereof (including but not limited to human subjects). In one embodiment, said subject has inflammation of the gastrointestinal tract. In one embodiment, said administering is oral (e.g. for treatment of inflammation in the gut). In one embodiment, said administering is by aerosol (e.g. for treatment of inflammation in the airways, including the lungs). In one embodiment, said administering is topical (e.g. for treatment of the skin). In a preferred embodiment, a higher local concentration of compound is achieved as compared to the concentration in the blood, regardless of the route of administration. In one embodiment, said subject has inflammation due to an autoimmune disease. In one embodiment, said autoimmune disease is inflammatory bowel disease. In one embodiment, said inflammatory bowel disease is selected from the group consisting of Crohn's disease and ulcerative colitis. In one embodiment, said subject has an inflammatory skin disorder. In one embodiment, said inflammatory skin disorder is atopic dermatitis. In one embodiment, said subject has an inflammatory lung disease. In one embodiment, said inflammatory lung disease is asthma.

In one embodiment, the present invention contemplates a compound (e.g. compound PI320, shown above) or a pharmaceutical composition, or a pharmaceutically acceptable salt thereof, for use in the manufacture of a medicament for reducing inflammation (e.g. in the intestinal tract, airways or on the surface of the body) in a subject having inflammation (including but not limited to human subjects).

In one embodiment, the present invention contemplates a method of treating overactive bladder syndrome comprising administering an effective amount of the compounds discussed herein, or a pharmaceutically acceptable salt thereof, to a subject in need thereof.

### Definitions

The term “alkyl” refers to a straight or branched hydrocarbon chain, containing the indicated number of carbon atoms. For example, C₁₋₁₂ alkyl indicates that the alkyl group may have from 1 to 12 (inclusive) carbon atoms, and C₁₋₄alkyl indicates that the alkyl group may have from 1 to 4 (inclusive) carbon atoms. In some embodiments, an alkyl group may be optionally substituted. Examples of C₁₋₄alkyl groups include methyl, ethyl, n-propyl, isopropyl, n-butyl, sec-butyl and tert-butyl. Unless otherwise stated, an alkyl is unsubstituted.

The term “cycloalkyl” as used herein refers to a saturated cyclic, bicyclic, tricyclic or polycyclic hydrocarbon groups having 3 to 12 carbons (e.g., 3, 4, 5, 6 or 7 carbon atoms). Any ring atom can be substituted (e.g., with one or more substituents). Cycloalkyl groups can contain fused rings. Fused rings are rings that share one or more common carbon atoms. Examples of monocyclic cycloalkyl groups include, but are not limited to, cyclopropyl, cyclobutyl, cyclopentyl, and cyclohexyl.

As used herein, “asthma” refers to a condition or disease that affects the airways of lungs. As an ex-ample, asthma typically results in episodes or attacks with symptoms including but not limited to breathlessness, chest tightness and wheezing.

In the context of treating a disorder, the term “effective amount” as used herein refers to an amount of the compound or a composition comprising the compound which is effective, upon single or multiple dose administrations to a subject, in treating a cell, alleviating, relieving or reducing one or more symptoms of the disorder in a subject. It is not necessary that the amount be limited to only those dosages which cure or completely eliminate one or more symptoms. An effective amount of the compound or composition may vary according to the application. In the context of treating a disorder, an effective amount may depend on factors such as the disease state, age, sex, and weight of the individual, and the ability of the compound to elicit a desired response in the individual. In an example, an effective amount of a compound is an amount that produces a pharmacologically useful change in a given parameter as compared to a control, such as in cells (e.g., a culture of cells) or a subject not treated with the compound. In one embodiment, a suitable dosage level is about 0.01 to 500 mg/kg per day, more typically about 1.0 to 250 mg/kg per day, still more typically about 5.0 to 100 mg/kg per day, or more typically 5.0 to 10 mg/kg per day. In one embodiment, the compound is administered orally, topically or aerosolized. In a preferred embodiment, a higher local concentration of compound is achieved as compared to the concentration in the blood, regardless of the route of administration.

A compound according to the present invention can be in the form of a salt, e.g., a pharmaceutically acceptable salt. The term “pharmaceutically acceptable salt” includes salts of the active compounds that are prepared with relatively nontoxic acids or bases, depending on the particular substituents found on the compounds. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulfonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts, alkaline earth metal salts, e.g. calcium or magnesium salts; and salts formed with suitable organic ligands, e.g. quaternary ammonium salts.

In another aspect, the invention provides pharmaceutical compositions comprising one or more compounds of this invention in association with a pharmaceutically acceptable carrier. Such compositions may be in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that compounds may be incorporated into transdermal patches designed to deliver the appropriate amount of the drug in a continuous fashion. For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture for a compound of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example, 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

As used herein, the term “subject” includes both animals and humans, including human patients in a medical facility.

As used herein, the term “topical” refers to administration of an agent or agents onto a body surface such as the skin.

As used herein, the term “Transdermal” refers to the delivery of an agent through the skin (e.g., so that at least some portion of the population of molecules reaches underlying layers of the skin).

Topical treatments can include, for example, liquid, gas, gaseous, gel, semi-solid, solid, particulate or aerosol, including in the form or a gel or powder. In one embodiment, drugs are applied topically (on top of the skin) or transdermally to the keratinocyte layer(s). In some embodiments, an agent may be applied using a brush, a pipet, a patch, or nebulizer, etc. In some embodiments, an agent applied topically results in transdermal delivery of an agent.

Compounds according to the present invention can be, for example, an enantiomerically enriched isomer of a stereoisomer described herein. Enantiomer, as used herein, refers to either of a pair of chemical compounds whose molecular structures have a mirror-image relationship to each other. For example, a compound may have an enantiomeric excess of at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99%.

Non-limiting examples of contemplated invention compound include the following structures which include some pairs of enantiomers:

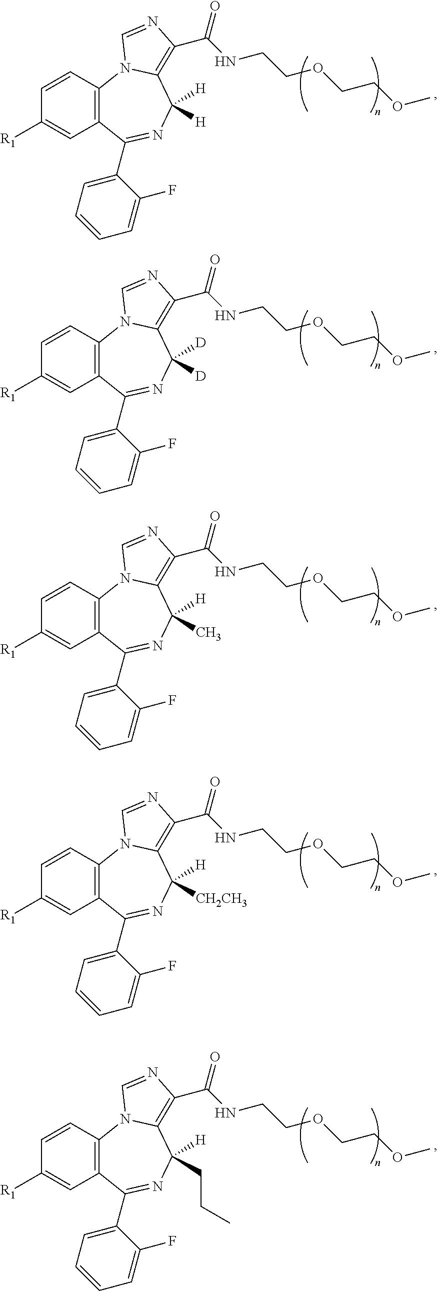

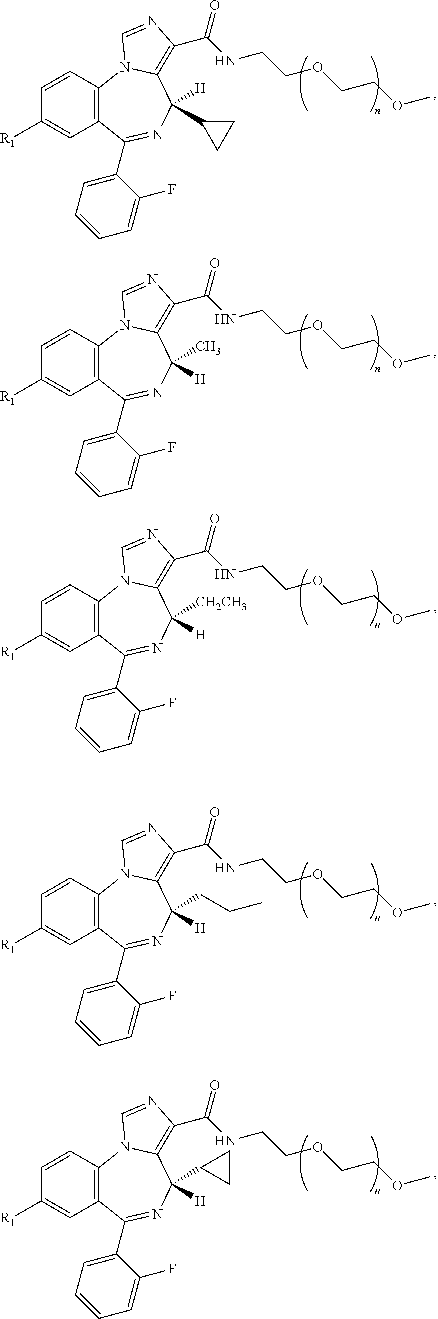

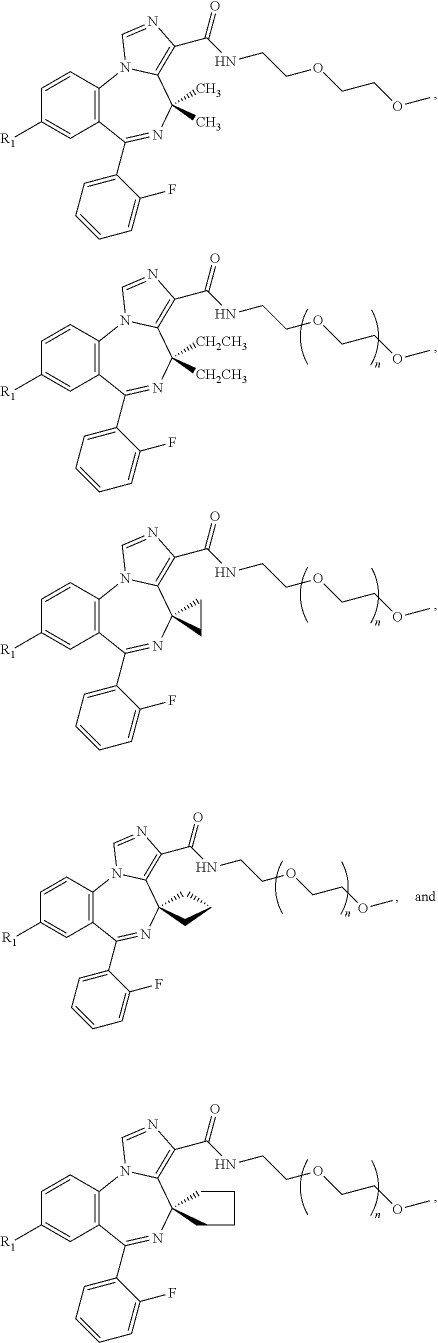

wherein: R₁ is H, Cl, Br, —OCH₃, —C≡CH, or cyclopropyl; and wherein n is any number between 1 and 15.

### BRIEF DESCRIPTION OF DRAWINGS

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1A shows exemplary change of bodyweight.

FIG. 1B shows exemplary change of consumption of 3% Dextran sodium sulfate (DSS); administered in order to induce ulcerative colitis.

FIG. 2A shows exemplary change of oxidized o-dianisidine was significantly different after a 15-minute incubation time.

FIG. 2B shows exemplary determination of colon MPO activity.

FIG. 3A shows exemplary comparative anatomy schematics of cross sections between healthy large intestine, an inflammatory large intestine, such as in Crohn's disease, that may involve the entire wall (mucosal to serosa layers) including muscle hypertrophy, an atypical cobblestone appearance of the epithelium, and may have damaged areas (i.e. loss of barrier function) as fissures that leak intestinal contents into the abdomen, and inflammatory large intestine limited to an inflammatory inner lining (mucosal region which may extend to the submucosal regions) such as in ulcerative colitis tissues (UC). For UC ulceration within the mucosa represented in the schematic, (i.e., loss of barrier function) may occur over time.

FIG. 3B shows an exemplary anatomy schematic of Crohn's Disease (CD) in a human small intestine with red-labeled regions corresponding to the areas that may be affected by each type of CD. Types of CD shown are, from top to bottom, left to right, ileocolitis, ileitis, gastroduodenal CD, jejunoileitis, Crohn's (granulomatous) colitis, and perianal CD. These types of CD and the areas that may be affected are further described below.

FIG. 3C shows an exemplary anatomy schematic of a small intestine showing regions of the stomach transitioning to the duodenum, jejunum, and ileum.

FIG. 3D shows an exemplary anatomy schematic of a human large intestines showing types of ulcerative colitis with yellow-, orange- and red-labeled regions corresponding to the areas affected by each type of colitis from the most limited to the most extensive, from left to right, top to bottom: proctitis (yellow region), proctosigmoiditis (orange region), distal colitis (orange region), extensive colitis (dark orange region) and pancolitis (red region) involving the entire large intestine.

FIG. 3E shows an exemplary anatomy schematic of a healthy human large intestine with labeled regions: transitioning from the ileum region of the small intestine through the ileocecal sphincter, into the cecum (where the appendix is located at the end of the cecum), ascending colon, transverse colon, descending colon, sigmoid colon, rectum, anal canal to the internal anal sphincter to the external anal sphincter of the anus.

FIG. 4 shows exemplary topical PI320 significantly reduces ear thickness (*=p≤0.05; **p≤0.01) compared to vehicle control.

FIG. 5 shows exemplary binding of PI310 and PI320 toward the GABA (A) receptors; structure shows an exemplary PI320.

FIG. 6 shows an exemplary docking study with PI310 and PI320. Overlay of docked conformations of PI310 (magenta) and PI320 (green) in the complex with the α₁β₃γ_{2L} GAB_{A}A receptor using structure 6HUO (Nature, 2019, 565:454-459) [2]. The α1⁺/γ2⁻ interface is indicated as α₁ (red) and γ₂ (cyan). Hydrogen and halogen bonds are indicated as dashed lines.

FIG. 7A shows the concentration-response curves for PI320 and DMSO controls. FIG. 7B shows percentage reduction of mean contractions by PI320 with respect to corresponding DMSO controls; *p<0.05, **p<0.01 vs DMSO. See FIG. 7A and FIG. 7B which show a concentration-dependent reduction of force with PI320.

### DESCRIPTION OF INVENTION

In one embodiment, the present invention provides a method of treating overactive bladder (OAB) syndrome a subject in need thereof (whether human or animal) by administering an effective amount of a compound (e.g. compound PI320, PI350, PI351, PI352 or variants thereof discussed herein), or a pharmaceutically acceptable salt thereof. In one embodiment, the present invention provides a method of reducing inflammation in a subject in need thereof, including but not limited to a subject having an autoimmune disease, by administering an effective amount of a compound (e.g. compound PI320, PI350, PI351, PI352 or variants thereof discussed herein), or a pharmaceutically acceptable salt thereof. In some embodiments, the autoimmune disease is selected from an Inflammatory Bowel Disease, an inflammatory lung disease, and an inflammatory skin disease. Descriptions of conditions contemplated to be treated with the compounds, (including inflammatory diseases) are provided below.

### I. General Inflammation in the Gastrointestinal System.

Inflammation in tissue of the gastrointestinal system has descriptive terms ranging from general terms to terms identifying specific regions. General disease terms include but are not limited to gastroenteritis, enteritis, colitis, etc., while specific diseased area designations include terms such as small intestinal ileitis, proctitis, etc. The following descriptions relate to general terms that may also refer to overlapping conditions or diseases.

### A. Gastroenteritis.

Gastroenteritis generally refers to irritation and inflammation anywhere in the digestive tract, i.e., involving the epithelial cell (keratinocyte) lining and underlying immune cells of the lamina propria. Gastroenteritis may be mild or severe. Mild gastroenteritis may result from, for example, indigestion or stress. As another example, a form of localized gastroenteritis in the stomach may be viral, also referred to as the stomach flu or the 24/48-hour bug. Gastroenteritis may also refer to life-threatening conditions resulting from food poisoning or a toxic ingestion, for example, after eating a substance that contains a toxin, such as a poisonous plant, mushroom or anthrax toxin. Gastroenteritis may also occur as the result of a disorder or disease, such as inflammatory bowel disease, irritable bowel syndrome, a side effect of medication, chemotherapy or radiation, as examples.

### B. Enteritis.

Enteritis generally refers to conditions arising from an initial irritation and inflammation of the small intestine (i.e., walls), usually accompanied by diarrhea. Enteritis may further involve other areas such as the stomach, and often further involves inflammation in the large intestine. Enteritis may also indicate or trigger the onset of an IBD (discussed below). Enteritis may also be caused by an autoimmune condition resulting in chronic inflammation, such as in Crohn's disease when inflammation is restricted to the small intestine.

Enteritis is typically caused by eating or drinking food items that are contaminated with bacteria, parasites, such as amoebae, or viruses. Pathogenic triggers typically settle in the small intestine and cause inflammation and swelling which may extend to the stomach and/or large intestine.

Enteritis may also be initiated by radiation, where radiation is an irritant resulting in radiation enteritis in the small intestine, where symptoms may occur during or shortly after radiation treatment. Radiation enteritis may be acute and/or chronic.

### II. Colitis.

Colitis is a general term referring to inflammation of the colon, i.e., large intestine, however colitis may also refer to disorders/diseases additionally associated with inflammation of the small intestine and other parts of the gastrointestinal system. Colitis may be acute, self-limited, or chronic, i.e., persistent. Colitis in humans is associated with intermittent, watery, diarrhea (with or without blood in the stool) and may include inflammation causing acute or chronic abdominal pain, cramping, and bloating. Additional symptoms depend upon the cause of colitis and may include fever, chills, fatigue, dehydration, eye inflammation, joint swelling, canker sores, and skin inflammation.

Colitis inflammation may be due to infection by virus, ameba, or a bacterium (such as Campylobacter) that produce toxins that irritate the lining of the intestine inducing inflammation. Colitis may also be caused by bacteria that directly infect the colon lining, i.e., mucosal region including epithelium.

Types of colitis include autoimmune colitis covering a range of inflammatory bowel disease (IBD) as a group of chronic colitides, ulcerative colitis (a chronic colitis that affects the large intestine), and Crohn's disease, a type of IBD that often leads to colitis and idiopathic inflammatory conditions. These last two types are described in separate sections under section III.

Colitis generally includes diseases in their inflammatory stages, such as enteritis, infectious colitis, Pseudomembranous colitis, necrotizing enterocolitis, ischemic colitis, acute mesenteric ischemia, radiation, allergic (response) colitis, several types of microscopic colitis, proctitis, and inflammatory bowel disease (IBD) (including Crohn's (colitis) disease, ulcerative colitis, etc.). Because different types of colitis in humans may have similar symptoms and overlapping causes, biopsies of human gastrointestinal tissue are frequently obtained.

### A. Necrotizing Enterocolitis (NEC).

Necrotizing Enterocolitis (NEC) refers to when portions of the inner lining, i.e., epithelium, of the large and/or small intestine, including an immature intestine, become inflamed then undergoes necrosis (tissue death). NEC is characterized by damage to the intestinal tract, ranging from mucosal injury to full-thickness necrosis and perforation. There is no one cause for NEC which is consider a multifactorial condition having risk factors that include premature birth and the presence of bacteria in an immature GI tract. As an example, NEC may occur after normal gut bacteria cause a local infection and inflammation by infecting the intestinal epithelium.

NEC is a common type of colitis in human newborns, premature, formula-fed infants, and may also be a condition in adults. In newborns, onset of NEC is typically during the first several weeks after birth, with the age of onset inversely related to gestational age at birth. In term infants, the reported median age of onset is 1-3 days, but onset may occur as late as age 1 month or more. There is also a form of adult necrotizing enterocolitis known by different local names (for instance, ‘Darmbrand’ in Germany and ‘pigbel’ in Papua New Guinea).

### B. Infectious Colitis.

Infectious colitis refers to when inflammation of the intestines is caused by infection of a pathogen (bacterial, parasitic, or viral). Infectious colitis is a common form of pediatric colitis and occurs in adults. Pathogens induce degeneration of the epithelium and inflammation of the lamina propria, even when the pathogenic organisms themselves do not penetrate to the lamina propria region.

### 1. Bacterial Colitis.

Bacterial colitis refers to colitis induced by bacteria. Examples of such bacteria include but are not limited to Escherichia coli (including both enterohemorrhagic E. coli [EHEC] and enteroinvasive E. coli [EIEC]) and species of Shigella, Salmonella, Campylobacter, Clostridium, Yersinia, including Yersinia enterocolitica, etc.

As an example, Salmonella infections can cause typhoid (enteric) fever or non-typhoid infections, which induce a significant proportion of food poisoning. Salmonella infections are typically spread via the fecal-oral route with outbreaks commonly associated with contaminated eggs, dairy products, and meats. Gastric acid is usually lethal to these bacteria, but susceptibility to infection is increased with decreased GI motility, rapid emptying of the stomach after gastrectomy, ingesting a large quantity of Salmonella bacteria, malnutrition, antibiotic use, and achlorhydria (lower levels of hydrochloric acid in gastric secretions). Salmonellae can penetrate the epithelial layer to the level of the lamina propria and evoke a leukocyte response in addition to producing several toxins.

Shigella species attach to binding sites on the surface of the intestinal mucosal cells. This organism may penetrate and proliferate inside of epithelial cells, which may lead to cell destruction, producing mucosal ulcerations, and bleeding. Shigellae also shed exotoxins that induce diarrhea.

E coli may include diarrhea in several different ways, depending on their specific pathologic characteristics. Pathologic strains of E. coli are classified as follows: Enteropathogenic; Enterotoxic; Enteroinvasive; Enteroaggregative; Enteroadherent; Enterohemorrhagic; and EHEC, including O157:H7 and O26:H11, which causes hemorrhagic colitis and systemic complications (e.g., hemolytic uremic syndrome [HUS]). The risk of developing HUS after infection with E. coli O157 is estimated to be 10-15% in children. In typical infectious colitis, the lamina propria of the large intestine is infiltrated by PMNs. EIEC, on the other hand, exhibits almost exactly the same pathogenetic mechanisms as Shigella.

2. Clostridium difficile Colitis.

A subtype of infectious bacterial colitis is Clostridium difficile colitis. C. difficile Colitis refers to inflammation of the colon associated with an overgrowth of the bacterium Clostridium difficile (C. diff). This overgrowth of C. difficile is most often related to recent antibiotic use but may be a result of other causes. C. difficile is typically associated with the presence of pseudomembranous and may also be referred to as antibiotic-associated colitis or C. difficile colitis.

### 3. Viral Colitis

Viral colitis refers to virally induced colitis. As an example, colitis may be caused by cytomegalovirus (CMV) infection, typically found in immunocompromised patients (e.g., organ recipients who are receiving immunosuppressive treatment). Viral colitis results in deep round ulcerations that have a tendency to bleed easily and profusely. Adenovirus infection can also cause a severe colitis in immunocompromised patients, especially those with AIDS, in addition to patients having solid organ and bone marrow transplants. Viruses include but are not limited to (Norwalk agent, Rotaviruses, cytomegalovirus [CMV], etc.).

As one example, Escherichia Coli (EC) induced inflammatory gastroenteritis. The common Escherichia coli strains carried in the human intestine have minimal or no invasive ability. HEC strains have acquired the genes to express Shiga-toxins. These toxins causes cell death, edema and hemorrhage in the lamina propria. The enteroinvasive E. coli (EIEC) has acquired certain genetic traits from Shigella sp. that allow it the same invasive capabilities that certain Shigella sp. possess. EHEC, the majority of the pathology occurs in the ascending and transverse colon lamina propria. Colonic biopsy specimens show focal necrosis and infiltration of neutrophils.

### 4. Parasitic Colitis.

Parasitic colitis refers to parasite-induced colitis, including but not limited to protozoan and non-protozoan parasites such as Giardia, Entamoeba, Balantidium coli, Cryptosporidium, Ascaris, etc. Chronic parasite-induced colitis may lead to UC. Moreover, parasite infections, thus inflammation, may be further found in the stomach and lungs.

As an example, Giardia lamblia, a flagellate, may colonize the small intestinal duodenum and jejunum where they adhere to the epithelium of the microvillus and induce mild pathologic changes. Shortening and thickening of the villi is associated with acute focal inflammatory changes in the mucosal epithelium and chronic inflammatory infiltrates in the lamina propria. Another example is amebiasis referring to a parasitic infection of the intestines caused by any of the amoebas of the Entamoeba group, including Entamoeba histolytica, or E. histolytica which is a common cause of parasitic colitis throughout the world. Transmission of Entamoeba spp. takes place through ingestion of trophozoites (referring to a growing stage in the life cycle capable of absorbing nutrients from a host), usually from water contamination, and person-to-person transmission (typically because of poor sanitation). Another example is Balantidium coli, a large ciliated protozoan that may cause colitis. Balantidiasis symptoms are similar to amebiasis.

As another example, common features in an intestinal Cryptosporidium infection include immune cell infiltration of the lamina propria, villus atrophy, crypt hyperplasia, a reduced barrier function (increased paracellular permeability), etc. Cryptosporidium frequently causes diarrhea due to inflammatory damage of the microvilli. The majority of human infections by Cryptosporidium are due to either Cryptosporidium hominis (C. hominis) and/or Cryptosporidium parvum (C. parvum).

### C. Pseudomembranous Colitis.

Pseudomembranous colitis refers to a form of inflammatory colitis characterized by the pathologic presence of pseudomembranes comprising of mucin, fibrin, necrotic cells, and polymorphonuclear leukocytes (PMNs). Thus, Pseudomembranous colitis generally refers to a non-specific histomorphologic description. Pseudomembranous colitis may also refer to colitis induced as antibiotic-associated colitis or Clostridium difficile colitis when these types of colitis further involve a pseudomembrane.

Factors that may increase risk of pseudomembranous colitis include: taking antibiotics; staying in the hospital or a nursing home; increasing risk along with an increase in age, and a higher risk in people over 65 years of age; having a weakened immune system; having a colon disease, such as inflammatory bowel disease or colorectal cancer; undergoing intestinal surgery; receiving chemotherapy treatment for cancer, etc. Pseudomembranous colitis may sometimes return, days or even weeks after apparently successful treatment for reducing inflammation. In relation to onset of antibiotic-associated pseudomembranous colitis, symptoms may begin as soon as one to two days after initiating an antibiotic treatment, or might take as long as several weeks after completing a course of antibiotic treatment.

D. Ischemic colitis.

Ischemic colitis (ischaemic colitis) refers to when inflammation and injury of the large intestine (ischemia) is triggered by inadequate blood supply or to a loss of blood supply to the colon (ischemia). Ischemia leads to mediator release, inflammation, and ultimately infarction. If a blood clot interrupts the flow of blood to a segment of the colon, the result is inflammation of that segment and, sometimes, even death [gangrene] of the segment). Although uncommon in the general population, ischemic colitis occurs with greater frequency in the elderly, and is the most common form of bowel ischemia.

Causes of the reduced blood flow can include changes in the systemic circulation (e.g., low blood pressure) or local factors such as constriction of blood vessels or a blood clot. However, in most cases, no specific cause can be identified.

Ischemic colitis is also a form of vasculitis that results from inflammation and ischemia of colonic mucosa, resulting in rectal bleeding and abdominal pain. This form of colitis is common in Henoch-Schönlein purpura (HSP), which is considered one of the collagen-vascular diseases.

### E. Allergic Colitis.

Allergic (response) colitis refers to an exaggerated response of the immune system, often to common substances such as foods causing inflammation of the intestine.

Allergic colitis refers to colitis resulting from an immune response to an allergen, for example, a hypersensitivity reaction to an allergen. One example of allergic colitis is a hypersensitive response to allergens in cow's milk or soymilk, as examples. Breast milk allergy refers to a food allergy induced in breastfed babies by heterologous proteins (typically cow's milk proteins) ingested by their mothers and appearing in their breast milk.

Immunologic responses including immune cells in the lamina propria, may range from allergic mast cell activation to more involved immune responses including mononuclear cells, leukocytes, lymphocytes, etc.

### F. Diversion Colitis.

Diversion colitis refers to an inflammation of the colon, which can occur as a complication of ileostomy or colostomy, often occurring within the year following the surgery. Ileostomy refers to a surgical operation in which a piece of the ileum is diverted to an artificial opening in the abdominal wall so-as-to bypass a damaged part of the small intestine. Colostomy refers to a surgical operation in which a piece of the colon is diverted to an artificial opening in the abdominal wall so-as-to bypass a damaged part of the colon. Diversion colitis frequently occurs when a neovagina is created by colovaginoplasty, with a varying delay in onset time after the original procedure. Colovaginoplasty, also known as a colon section, refers to an operation for creating a vagina by cutting away a section of the sigmoid (descending) or ascending colon and then using it to form a vaginal lining. Diversion proctitis colitis may also be induced by these types of surgery.

Despite the presence of a variable degree of inflammation in diversion colitis, which may include a diffuse increase in lymphocytes and plasma cells in the lamina propria, prominent lymphoid aggregates are observed histologically in biopsies of inflamed tissue. Inflamed areas may occur in remaining in-stream colon and/or in by-passed sections. In milder cases after ileostomy or colostomy, diversion colitis left untreated disappears naturally. In more severe cases, treatment is initiated, including but not limited to short-chain fatty acid irrigation, steroid enemas and mesalazine. Moreover, diversion colitis may trigger ulcerative colitis. Lim, et al., “Diversion colitis: a trigger for ulcerative colitis in the in-stream colon?” Gut 44:279-282 1999 [3]. Therefore, diversion colitis may be a risk factor for ulcerative colitis in predisposed individuals and that ulcerative colitis can be triggered by anatomically discontinuous inflammation elsewhere in the large intestine.

### G. Chemical Colitis.

Chemical colitis is a type of colitis, an inflammation of the large intestine or colon, caused by the introduction of chemicals to the colon. Chemical exposure may occur by an enema or other procedure, such as exposure to endoscope or colonoscopies cleaning solutions sometimes accidentally introduced into the colon during colonoscopy or other procedures. Endoscopically, chemical colitis can resemble ulcerative colitis, infectious colitis and/or pseudomembranous colitis, among others. Specific chemical exposure, such as during hydrogen peroxide enemas, common prior to 1950, soap enemas, glutaraldehyde, alcohol, radiocontrast dyes, etc. may result in chemical colitis.

Chemical colitis may trigger a flare of ulcerative colitis or Crohn's colitis.

### H. Microscopic Colitis.

Microscopic colitis refers to inflammation of the colon that is only visible when the colon's lining is examined under a microscope. The appearance of the inner colon lining in microscopic colitis is normal by visual inspection during colonoscopy or flexible sigmoidoscopy. The diagnosis of microscopic colitis is made when a doctor, while performing colonoscopy or flexible sigmoidoscopy, takes biopsies (small samples of tissue) of the normal-appearing lining from different regions of the colon during colonoscopy and then examines the biopsies under a microscope. The abnormalities of the colon's lining in microscopic colitis occur in a patchy distribution (areas of normal lining may coexist adjacent to areas of abnormal lining). For this reason, multiple biopsies should be taken from several different regions of the colon in order to accurately make a diagnosis.

The primary symptom of microscopic colitis is chronic, watery diarrhea likely caused by inflammation. There are two types of microscopic colitis: 1) lymphocytic colitis and 2) collagenous colitis. In lymphocytic colitis, there is an accumulation of lymphocytes (a type of white blood cell) within the lining of the colon. In collagenous colitis, there is an additional layer of collagen (scar tissue) just below the lining.

Lymphocytic colitis and collagenous colitis are contemplated to represent an autoimmune disorder similar to the autoimmune disorders that cause chronic ulcerative colitis and Crohn's disease. However, a previous study implicated long term (longer than 6 months) use of nonsteroidal anti-inflammatory drugs (NSAIDs) as a cause of microscopic colitis. In fact, some individuals' diarrhea improves after stopping the NSAIDs. Several other drugs have also been incriminated as a cause of microscopic colitis. The most common are proton pump inhibitors (PPIs) such as lansoprazole (Prevacid, Prevacid SoluTab), omeprazole (Prilosec, Zegerid), and esomeprazole (Nexium); the Statin simvastatin (Zocor); H2 blocker ranitidine (Zantac); SSRI sertraline Zoloft); and P2Y12 inhibitor ticlopidine (Tilcid).

Individuals with microscopic colitis can have diarrhea for months or years before the diagnosis is made. Typically, the symptoms begin very gradually and are intermittent in nature with periods when the person feels well, followed by bouts of chronic diarrhea. This chronic diarrhea of microscopic colitis is different from the acute diarrhea of infectious colitis, which typically lasts only days to weeks.

The patchy nature of microscopic colitis may be a reason why flexible sigmoidoscopy often is inadequate in diagnosing the condition because the abnormalities of microscopic colitis may be absent from the sigmoid colon (the colonic segment that is closest to the rectum and is within the reach of a sigmoidoscope) in some of the patients with microscopic colitis. Thus, biopsies of other regions of the colon accessible only with colonoscopy may be necessary for diagnosing microscopic colitis.

The long-term prognosis (course) of microscopic colitis is not clear. In approximately two-thirds of the patients with microscopic colitis, the diarrhea resolves spontaneously after several years. The remaining one-third of the patients with microscopic colitis experience persistent or intermittent diarrhea and/or abdominal pain for many years (possibly indefinitely) as there is no cure for the condition. This information came from: Microscopic Colitis (Lymphocytic Colitis and Collagenous Colitis) Medically Reviewed by a Doctor on Aug. 8, 2016 Medical Author: Bhupinder Anand, MD; Medical Editor: Jay W. Marks, MD.

Injury to the intestines can occur following radiation therapy for cancer. It can affect both the large and small intestines, is often progressive, and may lead to a variety of clinical consequences depending upon the extent of the injury. It usually develops three or more months after radiation therapy. Chronic radiation enteritis is due to an obliterative arteritis that leads to intestinal ischemia, which can result in stricture, ulceration, fibrosis, and occasionally fistula formation.

### I. Proctitis Colitis.

Proctitis colitis generally refers to chronic inflammation of the rectum. Proctitis has an acute (early) and chronic (late or slower) manifestation.

### J. Radiation Induced Colitis.

Adverse effects of radiation therapy is the development of inflammatory radiation colitis, radiation enteritis and radiation proctitis. Radiation colitis refers to colitis that develops after radiation, such as following treatment with radiation for prostate cancer. Radiation enteritis refers to irritation and inflammation of the large and small intestines. Radiation proctitis refers to inflammation and damage to the lower parts of the colon after exposure to x-rays or other ionizing radiation as a part of radiation therapy. Radiation enteritis has an acute (early) and chronic (later or slower) manifestation.

### K. Irritable (Inflammatory) Bowel Syndrome (IBS)

Irritable bowel syndrome (IBS), sometimes referred to as having “spastic colitis” may represent a mild inflammatory condition due to the presence of an inflammatory infiltrate in the lamina propria of the colonic mucosa, represented by increased numbers of T lymphocytes and mast cells compared with healthy subjects. Sinagra, et al., “Inflammation in irritable bowel syndrome: Myth or new treatment target?” World J Gastroenterol. 2 (7): 2242-2255, 2016 [4]. Infiltrates appear to be more predominant in the right than in the left colon (Salzmann, et al., “Morphometric study of colonic biopsies: a new method of estimating inflammatory diseases.” Lab Invest. 1989; 60:847-851) [5]. Also, individuals may have symptoms that mimic colitis such as diarrhea, abdominal pain, and mucus in stool. Nevertheless, the cause of symptoms in IBS is not clearly known.

### III. Inflammatory Bowel Diseases (IBD).

Inflammatory bowel disease (IBD) refers to chronic inflammation in human intestines causing swelling and irritation that may further involve other locations in the gastrointestinal tract, lungs and other parts of the body, such as joints, skin, etc. IBD symptoms are painful and lifelong as there is no cure for the majority of IBDs where current treatments might help to temporarily reduce symptoms. However, while IBD symptoms may be reduced for a time period, symptoms will arise again with flare-ups of inflammation.

The cause of IBD remains unknown in human patients but generally considered to be caused by environmental factors interacting with a genetically susceptible or physiologically susceptible human subject. Current research indicates that a human subject at risk of developing IBD likely involves a complex interaction of factors, including but not limited to antigens from the environment such as bacterial/pathogen exposure from food/water, in addition to the subject's susceptibility including a genetic predisposition to IBD (i.e. heredity, since IBD is familial), and physiological susceptibility which includes any one or more of the status of the person's immune system (because IBD involves abnormal immune regulation), damage to epithelium from pathogens, and constitution of resident gut bacteria, i.e. types and strains. While IBD can affect anyone, equally affecting males and females, a genetic predisposition to IBD is supported by the increased rates of IBD in northern European-Caucasians along with Jews of European descent (Ashkenazi Jews) which are currently more likely than other ethnic groups to have IBD. However, increasing rates of IBD are involving African Americans and Hispanics in the United States.

Studies have shown that 5% to 20% of affected individuals have a first-degree relative (parents, child, or sibling) with one of the diseases. A familial (hereditary) risk is greater with Crohn's disease than ulcerative colitis. The risk is also substantially higher when both parents have an IBD.

A genetic predisposition to IBD includes genetic effects on the immune system, etc. The lack of total concordance of disease among monozygotic twins, along with other differences, supports a role for environmental cofactors in the development of IBD.

What is known is that an inflammatory response, triggered in an area of the gastrointestinal tract is initiated by epithelial damage or by local immune cells, signals additional immune cells to migrate into the area as capillaries in the lamina propria expand, resulting in swelling and inflamed tissue. But instead of subsiding, as typical immune responses shut down over time while the area heals, the inflammatory condition of the inflamed area continues or increases over longer time periods, which causes a chronic long-term inflammation. Chronic inflammation may in turn cause thickening of the intestinal wall, ulceration, etc., and symptoms of an IBD patient, including pain and diarrhea.

One scenario-describing onset of IBD indicates that an intestinal inflammatory response is triggered by a foreign pathogen, i.e., bacteria, amoebae, or virus, via a toxin, contact with intestinal tissue or infection. This inflammatory response, instead of removing the pathogen or toxin while allowing healing of intestinal tissue then turning off, remains active which causes or allows additional damage to the local tissue. It is further contemplated that the immune cells stay active after the pathogen is removed because they begin reacting to normal gut bacteria, and/or local gastrointestinal cells, thus continuing and often spreading inflammation to larger areas of the gut. The immune cells are contemplated to remain abnormally active in part due to an underlying autoimmune genetic or physiological condition of the patient.

IBD symptoms may be constant or occur during flare-ups. General symptoms associated with IBD include an urgent need to move bowels; diarrhea; bloody diarrhea; or when areas of the intestine begin to block passage of their contents, constipation (leading to bowel obstruction); abdominal pain and cramping; a sensation of incomplete evacuation; weight loss; loss of appetite; nausea and vomiting; fatigue; etc. IBD symptoms may be reduced to some extent with simple dietary changes, such as switching to a diet that is low in fat; rich in fruits and vegetables; low in fiber and dairy products, and lifestyle changes such as reducing stress and resting, however as stated herein, there is no cure and symptoms will flare throughout the life of a patient.

Medications for IBD focus on reducing the swelling and/or irritation of the intestine. Medications include anti-inflammatory drugs; corticosteroids; immune system suppressors; antibiotics to kill germs in the intestinal tract; anti-diarrhea medication; laxatives; and pain relievers. Anti-inflammatoires, such as sulfasalazine (Azulfidine©), Mesalamine (e.g. AsacolC or Rowasa©), Olsalazine (Dipentum©), and Balsalazide (Colazal©), help reduce inflammation. Corticosteroids, such as prednisone (Deltasone©), have been shown to effectively reduce inflammation of the gastrointestinal tract in IBD patients. Medications, called immunosuppressant, have been used to treat IBD. Examples include Azathioprine (Imuran©), Mercaptopurine (Purinethol©), cyclosporine (e.g. Neoral© or Sandimmune©), and Infliximab (Remicade©). A fiber supplement, such as psyllium powder (Metamucil©) or methylcellulose (Citrucel©), may help relieve symptoms of mild to moderate diarrhea. Because inflammation may cause the intestines to narrow, resulting in constipation, as described herein, laxatives may be taken to relieve symptoms of constipation. Oral laxatives such as Correctol© have been used.

Acetaminophen (Tylenol©) may relieve mild pain. However, researchers have found a strong relationship between ingesting NSAIDs (nonsteroidal anti-inflammatory drugs), such as ibuprofen (Advil© or Motrin©) or naproxen (Aleve©), with IBD flare-ups.

While two common major types of IBD are well known, Crohn's disease and Ulcerative colitis, described below, IBD actually covers a range of gastrointestinal inflammatory diseases including Celiac disease, proctitis, ulcerative proctosigmoiditis; pancolitis and stomach ulcers. In fact, IBD may be associated with several different types of IBD and other autoimmune disorders such as celiac disease associated ulcerative colitis; dermatitis hepetiformis associated ulcerative colitis; systemic and discoid lupus associated ulcerative colitis; rheumatoid arthritis associated ulcerative colitis; ankylosing spondylitis associated ulcerative colitis; scleroderma associated ulcerative colitis; Sjogren's disease associated ulcerative colitis; porphyrinogenic drug induced ulcerative colitis, such as sulfasalazine; SLE associated with Crohn's disease; Crohn's disease of oral cavity; Crohn's disease of the hypopharynx; etc.

In some cases, IBD may be cured when detected early, such as for ulcerative colitis, early onset Crohn's disease, proctitis, and left sided colitis. However, typically there is no cure for IBDs.

The two major types of inflammatory bowel disease: ulcerative colitis (UC) and Crohn disease (CD), which are described in more detail below. The symptoms of these two illnesses may overlap. However, whereas Crohn's related inflammation typically starts in the small intestine and may spread to any area of the gastrointestinal tract (GI tract) as “skip lesions” where patches of diseased areas are separated by normal areas, UC is limited to inflammation in the colon. Crohn's disease can also affect the entire thickness of the bowel wall, from the mucosa to the adventia, unlike ulcerative colitis that mainly involves the innermost lining of the colon, the mucosa. When medical practitioners are not able to diagnose the specific type of IBD due to overlapping symptoms, the condition is called indeterminate colitis.

### A. Crohn's Disease.

Crohn's disease (CD) refers to a lifelong chronic inflammatory bowel disease of the gastrointestinal tract that arises in the small intestine that may progress to affect any part of the gastrointestinal system from the mouth to the anus. Crohn's is a chronic disease with patients experiencing time periods when the disease flares up and causes symptoms, followed by periods of remission when patients may not experience symptoms.

CD results in inflammation, ulcers, and bleeding in the digestive tract. It usually affects the end portion of the small intestine called the ileum. However, any part of the digestive tract can be affected, from the mouth to the anus. Crohn's associated inflammation most commonly affects the lower part of the small intestine (ileum) then may spread to or further involve the beginning of the colon. Crohn's related inflammation may further occur in any part of the large intestine, small intestine, or stomach. In fact, patches or lesions of CD related inflammation might occur anywhere in the GI system, including as ulcers or lesions in the oral cavity (mouth).

Further, CD inflammation may cause joint pain and swelling, inflammation and irritation of the eyes in addition to areas of painful, red and swollen skin, most often the legs. People with Crohn's disease often go through periods of flare-ups where they have severe symptoms and periods where their symptoms are more mild or non-existent. Someone with the disease who isn't displaying any symptoms is known to be in remission.

Crohn's disease may affect as many as 700,000 Americans. Men and Women are equally likely to be affected, and while the disease can occur at any age, Crohn's is more prevalent among adolescents and young adults between the ages of 15 and 35. People of Jewish heritage are more likely to get Crohn's disease. Risk may also be increased if you have family members with inflammatory bowel disease or other autoimmune diseases.

The cause of Crohn's disease is not known and there is no known cure for Crohn's disease. The environment also appears to play a role as Crohn's is more common in developed countries than in undeveloped countries, and occurs in more people in urban rather than in rural areas, and occurs in more people of northern rather than southern climates. Diet and stress may aggravate Crohn's Disease, but they do not cause the disease on their own. People suffering from Crohn's often experience loss of appetite, may lose weight, and have a feeling of low energy and fatigue. Among younger children, CD may delay growth and development.

Chronic Crohn's disease inflammation in the intestines can cause the walls of digestive organs to thicken or form scar tissue. This wall thickening from inflammation can narrow a section of intestine, i.e., stricture, which may lead to an intestinal blockage. A stricture refers to a narrowing of a section of intestine that, in turn, causes problems by slowing or blocking the movement of food through the area. Nausea and vomiting or constipation may be signs of a stricture, which may lead to hospitalization and also to surgery to correct it. Crohn's disease can disrupt the normal function of the bowel in a number of ways such as when the bowel tissue may: swell, thicken, or form scar tissue, leading to blockage of the passageway inside the bowel; develop ulcers that can involve the deep layers of the bowel wall; lose its ability to absorb nutrients from digested foods, a condition called malabsorption; develop abnormal passageways known as fistulas from one part of the bowel to another part of the bowel, or from the bowel to nearby tissues such as the bladder or vagina. In severe cases, Crohn's can lead to tears (fissures) in the lining of the anus, which may cause pain and bleeding, especially during bowel movements. Inflammation may also cause a fistula to develop. A fistula is a tunnel that leads from one loop of intestine to another, or that connects the intestine to the bladder, vagina, or skin. This is a serious condition that requires immediate medical attention.

Symptoms include but are not limited to: diarrhea; abdominal cramps and pain; rectal bleeding; weight loss; fatigue, weakness; nausea; fever; mouth sores; sores, abscesses in the anal area. Complications of untreated Crohn's disease may lead to: Fistulas, i.e., abnormal connections between the intestine and other organs or tissues, such as the bladder, vagina, or skin; intestinal obstruction; liver disease; bowel perforation; bleeding; kidney stones; gallstones; osteoporosis, etc. Extraintestinal manifestations, which are slightly more common in CD than in UC, result from bacterial products and inflammatory mediators (e.g., cytokines, prostaglandins, and reactive oxygen metabolites) entering and subsequently being deposited in various tissues and organs, such as the eyes (uveitis), skin (erythema nodosum), liver (cholangitis, hepatitis), and joints (arthritis).

Treatment may include dietary changes and/or medications to reduce symptoms. Dietary changes include avoid foods that trigger symptoms, which may be dairy foods if the patient also has lactose intolerance; highly seasoned foods; and high-fiber foods. However, these foods are different for each person. There are many types of medications that are used to treat Crohn's disease however many of the treated patients continue to experience symptoms. Examples of these medications include: Aminosalicylate medications, such as sulfasalazine, mesalamine, and olsalazine; Anti-inflammatory medications, such as prednisone, methylprednisolone, and budesonide; Immune modifiers, such as azathioprine, 6-mercaptopurine, and methotrexate; TNF inhibitors, such as infliximab, adalimumab, and certolizumab; and antibiotics, such as metronidazole, ampicillin, and ciprofloxacin.

Severe Crohn's may not improve with medications such that for certain patients the diseased section of the intestine removed. The two remaining healthier ends of the intestine are then joined together, i.e., re-sectioned. However, there remains a high risk for the disease occurring in the remaining “healthy” tissue. Surgery may also be done to remove obstructions or repair/close fistulas. Approximately 70% of children with CD require surgery within 10-20 years after the diagnosis.

There are at least six types of Crohn's disease. The following are brief descriptions of types located in the small intestine, large intestine and a part of the stomach.

### 1. Crohn's Ileitis.

Human patients with ileitis in general have inflammation of the ileum. Crohn's Ileitis is a chronic ileitis inflammation affecting the ileum, the third portion of the small intestine, between the jejunum and the cecum.

Patients experience considerable weight loss, diarrhea, and cramping or pain in the middle or lower right part of the abdomen, similar to symptoms of ileocolitis, see below, which further involves the beginning of the colon. In addition to the inflammatory intestines, fistulas (an abnormal connection between two hollow spaces (i.e., two epithelialized surfaces), such as intestines, blood vessels, or other hollow organs), or inflammatory abscesses (a collection of pus that accumulates within the tissue due to an inflammatory reaction) may also form in the lower right section of the abdomen where the ileum is located.

2. Crohn enterocolitis (or ileocolitis).

Ileocolitis is a common type of Crohn's disease. It affects the small intestine in the area of the ileum, at the end of the small intestine, and the beginning of colon (in the area of the cecum-appendix/ascending colon). Human patients who have ileocolitis experience considerable weight loss, diarrhea, and cramping or pain in the middle or lower right part of the abdomen.

### 3. Gastroduodenal Crohn's Disease.

This form of Crohn's disease involves both the stomach (typically the pyloric area) and duodenum of the small intestine, which is the first part of the small intestine located after the pyloric area of the stomach. People with this type of Crohn's disease suffer nausea, weight loss, and loss of appetite. In addition, if the narrow segments of bowel are obstructed, they experience vomiting.

### 4. Crohn's Jejunoileitis.

This form of the disease affects the upper half of the small intestine, i.e., jejunum. It causes areas of inflammation. Symptoms include cramps after meals, the formation of fistulas, diarrhea, and abdominal pain that can become intense.

5. Crohn's (granulomatous) Colitis.

This form of Crohn's disease involves merely any area of the colon, rectum or anus. Symptoms include skin lesions, joint pains, diarrhea, rectal bleeding, and around the anus, the formation of ulcers, fistulas, and abscesses. Skin lesions and joint pains are more common in this form of Crohn's than in others.

### 6. Perianal Crohn's.

Perianal Crohn's refers to inflammation around the anus.

### B. Ulcerative Colitis.

Ulcerative colitis (UC) refers to a long-term form of a chronic inflammatory bowel disease arising in the colon and confined (limited) to the mucosa. Thus ulceration is generally shallow and does not extend into muscularis propria unlike Crohn's disease. UC disease limited to the rectum refers to ulcerative proctitis (colitis). UC beginning in the rectum may spread proximally through the large intestine, typically without skipping Haustra sections (segments).

Ulcerative colitis symptoms may come and go. Remission can last for months or years, but the symptoms eventually return. Not knowing when symptoms will flare can add to the stress of the disease and make it difficult to come up with an effective treatment plan.

Ulcerative colitis sometimes causes complications that require hospitalization. These may include an ulcer that is bleeding profusely or severe diarrhea that causes dehydration. If there is a tear in the colon, it may need to be surgically repaired. For people with severe ulcerative colitis, a surgery to remove the colon may be done.

The symptoms of ulcerative colitis may include: Diarrhea or rectal urgency. Some people may have diarrhea 10 to 20 times a day. The disease usually causes bloody diarrhea and mucus.

With UC, small ulcers can develop on the colon's lining (mucosa) producing pus and mucus. This can cause abdominal discomfort and frequent emptying of the colon (diarrhea). Around 50% of people with UC are diagnosed with ulcerative proctitis or proctosigmoiditis.

UC is referred to as an autoimmune disorder. There is no known cure for UC. Some people have surgery to remove parts of their colon and rectum that are affected but inflammation may arise if any of the colon, rectum or anus are left in the patient. Often surgery doesn't remove symptoms and complications of IBD.

### 1. Pancolitis/Pan-ulcerative Colitis/Universal Colitis.

Pancolitis/Pan-ulcerative colitis/Universal Colitis refers to a severe form of a life-long duration ulcerative colitis that spreads through the entire large intestine, normally stops abruptly at the ileocecal valve, however in some cases distal ileitis may occur. The appendix and appendiceal orifice may also be involved.

This form of ulcerative colitis is spread throughout the entire large intestine including extending proximally to the splenic flexure, the cecum, right colon, the left colon, the transverse colon and the rectum. Twenty percent of people start with another form of UC, such as proctitis, proctosigmoiditis or left-sided colitis, however over time the inflammation spreads throughout their colon resulting in pan-ulcerative colitis. Symptoms of pancolitis include bloody diarrhea, abdominal pain and cramps, weight loss, fatigue, fever, and night sweats. There is no known cause of UC.

UC may also lead to cancer, where risk factors for developing adenocarcinoma in UC are the duration and extent of disease. After the first decade of disease, the risk of development of colon cancer increases rapidly. Colectomy may be performed if there is finding of high-grade dysplasia viewed by colonoscopy or in a biopsy.

The course of UC is marked by remissions and exacerbations. Most patients respond initially to medical treatment, and many children with mild manifestations stay in remission on prophylactic therapy, for example with 5-aminosalicylic acid (5-ASA). About 70% children with UC enter remission within 3 months of initial therapy with 50% remaining in remission over the next year. Colectomy within 5 years may be required in as many as 26% of children who present with severe disease compared with less than 10% of those who present with mild disease.

In biopsies of inflamed areas, an inflammatory infiltrate may be observed, indicative of chronic inflammation, in the lamina propria and submucosa, comprising lymphocytes, plasma cells, eosinophils; basal lymphoid aggregates may be present with few granulomas. A neutrophilic infiltrate is typically present when disease inflammation is active, involving epithelium of surface and crypts with frequently observed crypt abscesses. Lamina propria fibrosis may be present.

### 2. Proctosigmoiditis.

Proctosigmoiditis refers to a form of ulcerative colitis that affects the rectum and sigmoid colon (the S-shaped last part of the large intestine, leading into the rectum). Treatments currently include medication and surgery. Some people have severely inflamed or damaged parts of their colon surgically removed. This can reduce or eliminate the symptoms of proctosigmoiditis, however it does not get rid of the disease and there is a risk that it will return to another area of the colon in the future.

### 3. Left-Sided Colitis.

Left-sided colitis (also known as distal colitis) refers to inflammation that begins at the rectum and extends as far as a bend in the colon near the spleen called the splenic flexure.

### C. Indeterminate Colitis.

Indeterminate colitis is a general term referring to a chronic idiopathic (i.e., unknown cause) type of colitis that cannot be separated as either Crohn colitis or ulcerative colitis by a medical practitioner using conventional diagnostic modalities.

Iv. Overactive Bladder (OAB) Syndrome

Overactive bladder (OAB) syndrome, including interstitial cystitis (IC), is characterized in patients by the presence of urinary urgency in humans and abnormally frequent urination in animals. In fact, OAB is a common condition in mammals, such as humans, dogs, cats, etc., Some mammals having OAB are used as animal models for testing treatments, including rabbits, mice, rats, nonhuman primates, etc., In human patients, OAB results in a frequent and often sudden urge to urinate that may be difficult to control. A patient may feel the need to pass urine many times during the day and night, and may also experience unintentional loss of urine immediately after an urgent need to urinate (urgency incontinence). An OAB patient may urinate frequently, usually eight or more times in 24 hours and/or wake up more than two times in the night to urinate (nocturia). Even when the patient is able to get to the toilet in time, after sensing an urge to urinate, unexpected frequent urination and nighttime urination can be disruptive to life. OAB may be related to interstitial cystitis/painful bladder syndrome (IC/PBS). In fact, urgency, frequency, and nocturia are common symptoms of both OAB and IC.

A normal bladder is controlled by the central nervous system. The smooth muscles (i.e. the detrusor muscle, i.e. bladder, and the internal urethral sphincter) are controlled by the two different divisions of the autonomic nervous system. The parasympathetic nervous system stimulates the detrusor muscle to contract, causing a mammal to urinate. The sympathetic nervous system stimulates contraction of the internal urethral sphincter, causing the mammal to hold urine. Somatic efferent neurons stimulate contraction of the external urethral sphincter, which is skeletal muscle. As the bladder fills, nerve signals sent from the bladder to the brain eventually trigger a feeling of the need to urinate. Normal bladder function involves coordination of afferent signals originating from the bladder wall combined with excitatory or inhibitory signals from the brain to provide a neurological appropriateness to urinate that is ultimately under conscious control by the prefrontal cortex. For urinating, these nerve signals coordinate the relaxation of the pelvic floor muscles and the muscles of the urethra (urinary sphincter muscles) while the muscles of the bladder tighten (contract), pushing the urine out through the urethra.

An overactive bladder happens when bladder neurons and/or bladder muscles have spontaneously activity causing bladder muscles to expel urine, even when the volume of urine in the bladder is low.

Chronic inflammation, including as a subtype of neurogenic inflammation, may be involved with patients having an OAB, including cystitis patients. Noninfectious induced inflammation may occur in association with other diseases such as, gynecological cancer, PID, and Crohn's disease.

### V. Immunology of Mucosa and Gut.

Mucosa associated lymphoid tissue (MALT) refers to lymphoid tissue and lymphocytes located in the mucosal epithelial cell layer and lamina propria throughout the body. According to their location, lymphocytes are subdivided into intraepithelial lymphocytes (IEL), lamina propria lymphocytes and lymphocytes organized in follicles in association with epithelial cells (e.g., subepithelial lymphoid follicles). The latter may extend into the muscularis mucosae layer. MALT is present in the gastrointestinal system.

Intraepithelial lymphocytes (IEL) refer to lymphocytes found in the epithelial layer and are present in-between the epithelial cells lining the surface, e.g., CD8+T lymphocytes.

Mucosal lymphoid follicles, which in aggregates of on average 30-50 follicles are called Peyer's patches, contain mononuclear cells, T cells, including CD43+, CD8, γδ+ T cell receptor, B cells, plasma cells, etc. In other words, isolated or aggregated lymphoid follicles forming Peyer's patches (PPs) may be found in areas of the intestine. PPs are considered immune sensors of the intestine by their ability to transport luminal antigens and bacteria and induction of immune tolerance or defense against pathogens resulting from a complex interplay between immune cells located in the lymphoid follicles and the follicle-associated epithelium. The M cell refers to a specialized epithelial cell that transports luminal antigens, thus allowing access to immunocompetent cells. It plays a role in mucosal-based immunity and antigen tolerance.

In healthy gastrointestinal tissues, T lymphocytes are found at mucosal surfaces, primarily in the epithelium, the lamina propria cores of the villi, Peyer's patches and lymphoid follicles. Less common in normal colon epithelium and lamina propria but abundant in isolated colonic lymphoid follicles. When present, lymphoid follicles might be visible at microscopic examination of biopsies, such as a biopsy of the ileum.

Follicle-associated epithelium (FAE) refers to areas covering the Peyer's patches. The specialized epithelium overlying lymphoid aggregates, i.e., FAE, is distinct from the surrounding villous epithelial surfaces. It characteristically has fewer goblet cells and contains membranous cells or M cells.

Gut associated lymphoid tissue (GALT) specifically refers to mucosal lymphoid tissue and lymphocytes located in the gastrointestinal system. GALT tissue includes lamina propria lymphocytes (LPL), intraepithelial lymphocytes (IEL), Peyer's patches and scattered follicles.

GALT is especially prominent in the appendix and terminal ileum where it forms Peyer's patches along the anti-mesenteric border. Four compartments are distinguished in Peyer's patches, which refer to small masses of lymphatic tissue found throughout the ileum region of the small intestine. Peyer's patches appear histologically as oval or round lymphoid follicles or nodules (similar to lymph nodes) located in the lamina propria layer of the mucosa and may extend into the submucosa of the ileum. Smaller lymphoid nodules can be found throughout the intestinal tract.

### VI. Lamina Propria of the Gastrointestinal System.

Lamina propria refers to a layer of loose connective tissue that extends from the subepithelial basement membrane complex (underneath epithelial cells) to the muscularis mucosae in tubes found in humans, e.g., gastrointestinal. For example, in the gastrointestinal tract, LP is found between villi of the stomach, small intestine and large intestine, typically separated from the epithelial cells by a basement membrane.

Healthy lamina propria in vivo (e.g., non-inflamed in humans without an inflammatory disorder) provides immunological cells, nutritional support for the epithelium, e.g., from capillaries, and structural support for the epithelium e.g., by connective tissue secreting cells. Thus, lamina propria contains a variety of cell types including lymphocytes and other immune cells of MALT/GALT. In particular, healthy lamina propria generally comprises stromal cells, extracellular matrix, fibroblast cells, immune cells including various types of leukocytes, such as mononuclear cells, lymphocytes, B cells, T cells, natural killer cells, plasma cells, macrophages, eosinophils, and mast cells, along with capillary endothelium, etc.

Lamina propria includes capillary beds, wherein the healthy (non-inflamed) capillaries are lined with a single layer of endothelial cells. In the small intestine, lamina propria of villi includes lacteals (i.e., lymphatic capillaries) in addition to smooth muscle fiber cells. Where epithelial invaginations are densely packed (e.g., gastric glands of stomach), lamina propria can be relatively inconspicuous, i.e., by histology observation and thus contains few cells. Where the mucosal epithelium is extensively evaginated (e.g., intestinal villi) or invaginated (intestinal crypts), the location of lamina propria “beneath” the epithelium amounts to filling-in the spaces between nearby epithelial surfaces (i.e., surrounding each crypt, within each villus).

The lamina propria that surrounds crypts in healthy colon tissue contains eosinophils, lymphocytes, plasma cells, and some histiocytes. Relative to the left colon and rectum, the right colon contains greater numbers of immune cells in the lamina propria, including more plasma cells and eosinophils. In fact, areas around the ileocecal valve appears to have inflamed the lamina propria in healthy tissue merely because of the number of cells present. The left side of the colon contains significantly fewer cells within the lamina propria, and the surface epithelium contains more goblet cells and fewer absorptive cells relative to the right colon. Cerilli and Greenson, (2012) The Differential Diagnosis of Colitis in Endoscopic Biopsy Specimens: A Review Article. Archives of Pathology & Laboratory Medicine: Vol. 136, No. 8, pp. 854-864 [6].

A subtype of leukocytes in the lamina propria are the cells of the monocyte/macrophage lineage. In the colon they are diffusely present in the subepithelial part of the lamina propria. They are a heterogeneous group composed of cells having more phagocytic properties and cells equipped for antigen presentation. They appear often as foamy histiocytes. Other myeloid cells that normally reside in the lamina propria are eosinophils and mast cells. Neutrophils are not typically present in healthy lamina propria. Fibroblasts are located randomly, distributed throughout the lamina propria and in the most superficial portion and the pericryptal fibroblast sheet, tightly opposed to the subepithelial basement membrane complex.

Mesenchymal derived cells are also found in intestinal lamina propria including intestinal stromal cells (e.g., myofibroblasts and fibroblasts), mural cells (pericytes) of the vasculature (also part of the intestinal stromal cells), bone marrow-derived stromal stem cells, smooth muscle of the muscularis mucosae, and the smooth muscle of the small intestinal villus core surrounding the lymphatic lacteals. In fact, myofibroblasts are considered nonprofessional immune cells that may be an alarm system for the gut and as a participant in peripheral immune tolerance (Powell, et al., “Mesenchymal Cells of the Intestinal Lamina Propria.” Annual Review of Physiology, Vol. 73:213-237 (Volume publication date March 2011), First published online as a Review in Advance on Nov. 3, 2010) [7].

### VII. Asthma, Airways and Respiratory Viruses.

For reference, information is provided below on asthma, airway anatomy in general in relation to asthma and respiratory viruses.

### A. Asthma.

Asthma refers to a chronic and often a lifelong respiratory disease, ranging from mildly irritating to a serious, even life-threatening condition, termed severe asthma or ‘Status Asthmaticus’. In other words, asthma symptoms vary in frequency and severity. When a person is affected with asthma, in general it becomes more difficult to move air in and out of the lungs. Asthma can start at any age but it most commonly starts in childhood. At least 1 in 10 children and 1 in 20 adults have asthma.

Although asthma makes breathing difficult for millions, including children, there is no cure. In fact, nearly 26 million Americans have asthma, including more than 7 million children. In America, it causes millions of lost school and workdays every year and is the third leading cause of hospitalization among children.

The airways' epithelial lining in an asthma patient tends to be in a hypersensitive state often characterized by redness and swelling (inflammation), that may be referred to as ‘asthmatic airways’. This hypersensitive state makes the airways react to an asthma “trigger(s)” such that these asthmatic airways are extra sensitive to certain compounds in the environment, such as dust, chemicals, smoke, pet dander, etc., and/or sensitive to mere exposure to an environmental condition such as cold air, misty air, evening air or time period, etc., alone, or in combination with an increased breathing rate, such as caused by stress or exercise.

When a person breathes in a trigger, the inside linings of the airways (epithelial lining) swell such that asthma usually causes episodes of breathlessness that may be accompanied by chest tightness and/or wheezing. A trigger may also induce bronchial spasms where the muscles that wrap around your airways tighten, making breathing even harder. Spasms of the bronchial tube narrow the space for the air to move in and out of the lungs. When this happens, it's referred to as an asthma flare-up, asthma episode or asthma “attack.” Common symptoms are coughing, wheezing, breathless, and may develop a feeling of chest tightness. Symptoms can range from mild to severe between different people, and at different times in the same person. Each episode of symptoms may last just an hour or so, or persist for days or weeks unless treated.

Asthma attacks often do not stop on their own without asthma treatment. When early warning signs of an asthma attack are ignored, this increases the risk of developing Status Asthmaticus, which may require immediate medical attention and treatment, including hospitalization.

The severity of the asthma attack may depend on how well the underlying asthmatic airways are controlled, i.e., reflecting how well the airway inflammation is being controlled. Inhaled steroids (e.g., albuterol) are potent anti-inflammatory drugs that are highly effective in reducing inflammation associated with asthma. However, some asthma attacks do not respond to immediate care (quick-relief medications), e.g., a bronchodilator inhaler rendering an acute asthma attack potentially life-threatening. When an acute asthma attack is unresponsive to treatment with an asthma inhaler (e.g., albuterol), this may be a symptom of a severe asthma attack.

Symptoms of a severe asthma attack may also include: persistent shortness of breath; the inability to speak in full sentences; breathlessness even while lying down; chest that feels closed; bluish tint to lips; agitation, confusion, or an inability to concentrate; hunched shoulders and strained abdominal and neck muscles; and a need to sit or stand up to breathe more easily. These symptoms may also be signs of an impending respiratory system failure from a lack of oxygen due to obstructed airways, which requires immediate medical attention. Ensuing respiratory failure results in hypoxia, carbon dioxide retention and acidosis.

Other physical asthma related symptoms might be noticed as inflammation within the mouth, pharynx, and upper airway, along with increased mucus production and narrowed airway openings. Asthmatic conditions include smaller airways, including bronchioles, in addition to the larger bronchial tubes. For reference, an overview of the “airway” or “respiratory” tract is provided below.

### B. Airway (Respiratory) Tract.

A human respiratory tract or “airway” refers to a pathway that carries air from the outside of the body to the lungs and then back out. For reference, it can be divided into upper (conducting) and lower (conducting and respiratory) parts. The upper respiratory tract includes the nose, sinuses, throat (pharynx) and voice box (larynx). After passing through this upper conducting region, inhaled air passes through the lower respiratory tract, including the portion of the larynx below the vocal cords, single trachea, then highly branched conducting airways of bronchi and bronchioles. More specifically, the single trachea divides into a right main bronchus and left main bronchus (bronchus: singular). As the air travels to the lung tissue, from larger to smaller airways, it moves through the trachea into primary bronchi (bronchi: plural), secondary bronchi (lobar bronchi), tertiary bronchia (segmental bronchia) then bronchioles. Intrasegmental bronchi refer to bronchial branches within the lung tissue contiguous with bronchioles. The bronchioles terminate into small collections of air sacs known as alveoli, which is where the actual exchange of CO2 and Oxygen occur. Thus, bronchioles include terminal bronchioles, each which support an airway into a lobule of air sacs, and respiratory bronchioles with attached alveolar sacs. Each respiratory bronchiole supplies an airway into each air sac (acinus or respiratory unit). Thus, respiratory parts of the airway include respiratory bronchioles contiguous with an alveolar air duct, allowing air to move from the respiratory bronchioles into and out of the alveolar sac.

In other words, as “airways” of the lungs, inhaled air passes through the nose or mouth into the larynx, then into the trachea through bronchi into bronchioles then into the alveoli (air sacs) of the lungs where an alveolar-capillary interface allows CO₂ and O₂ air-red blood cell exchange.

Primary bronchi are located in the upper portion of the lungs, with secondary bronchi near the center of the lungs. Tertiary bronchi are located just above the bronchioles. Structurally, the intrapulmonary (secondary) bronchi have a lining of pseudostratified ciliated columnar epithelium, a basement membrane with a lamina propria containing an abundant longitudinal network of elastic fibers made by resident stromal cells.

Stomal cells in the respiratory airway linings, i.e., connective tissue cells, are cells secreting connective tissue compounds, include but are not limited to fibroblast cells, pericyte cells, etc. Stromal cells particularly in the respiratory tract contribute elastic connective tissue. Additionally, there are spirally arranged bundles of smooth muscle, abundant mucoserous glands, and, in the outer part of the wall (adjacent to bodily tissues), and irregular plates of supportive hyaline cartilage.

In bronchi, the C-shaped cartilage of the trachea is replaced by separate plates of cartilage. At the same time, the lamina propria becomes surrounded by a band of smooth muscle fibers that are arranged spirally and crisscross one another. The smooth muscle can be considered as a separate layer, the muscularis, lying between the mucosa on the one side and the submucosa, fibrocartilage plates and adventitia on the other side.

The mucosa of bronchi, as in the trachea, has epithelium (e.g., ciliated pseudostratified columnar with goblet cells, a basement membrane, and a lamina propria. Goblet cells in bronchi and bronchiole are less numerous than in the trachea. They are usually filled with mucous secretory droplets, which are discharged into the lumen where they form a mucous blanket on top (apical) surface of the epithelial cells. Goblet cell mucus is contemplated to effect ciliary action of the epithelial cells. For example, in the absence of mucus, cilia fail to continue a beat-like motion. This movement is restored by the addition of mucus in experimental systems.

Serous and mucous glands are present in the submucosa of bronchi, with decreasing numbers per area with each division of the branches into smaller order bronchi. The adventitia or peribronchial layer contains many elastic fibers and separates the wall of the bronchus from pulmonary parenchyma. It permits bronchi to move independent of other lung parenchyma.

In contrast to bronchi, bronchioles lack cartilage and glands and generally have a diameter less than 1 mm. Three layers can be distinguished: mucosa, muscularis, and an outer layer. Due to the absence of cartilage and contraction of smooth muscle the mucosa is highly folded. It is lined with a simple cuboidal epithelium, which besides ciliated and mucous-secreting cells includes Clara cells.

The muscularis is the thickest layer. It has thick bands of smooth muscle, which completely encircle the bronchiole. The connective tissue of the thin outer layer is continuous with the parenchyma of the lung so that these bronchiole passages move with the lungs.

When the bronchi become swollen due to irritants or infection, bronchitis may result making breathing more difficult. Bronchitis sufferers also tend to have much more mucus and phlegm than someone without inflamed bronchi. Asthma is a condition that typically affects the smaller airways, e.g., bronchioles. This area of the airway may narrow (i.e., constrict) due to a bronchospasm.

### C. Cystic Fibrosis.

Cystic Fibrosis (CF) refers to an inherited disorder that may cause severe damage to the lungs, digestive system (including intestine) and other organs in the body. In people with cystic fibrosis, secretions from goblet cells, or equivalents, are increased in amount and become sticky and thick instead of the healthier slippery and thin secretions. Secretions in CF patients may plug up drainage tubes, airway ducts and passageways, especially in the lungs, instead of acting as thin and slippery lubricants for moving microbes away from cells and out of the body. Furthermore, sticky and thick mucus in the lungs traps microbes and provides nutrients for their replication and growth unlike healthy and thin mucus. Thus, examples of commonly associated complication in CF patients are extreme breathing difficulties, which may lead to susceptibility for chronic respiratory infections, as well as defective signaling in the intestine.

At least one genetic change (i.e., mutation) between the cftr (chloride channel CFTR (cystic fibrosis transmembrane conductance regulator)) gene of nonCF patients vs. CF patients is associated with differences in controlling movement of salt and water in and out of cells. In CF patients, the protein of this gene does not function as well as in healthy patients, in part, causing the thick and sticky mucus, along with increased salt in sweat. A patient may be a CF carrier, having one allele with a cftr mutation, or display active CF with both alleles having a cftr mutation.

Bacterial infections (e.g., Pseudomonas aeruginosa) on the disease (CF) background are found clinically. In fact, a chronic infection with P. aeruginosa is one of the main proven perpetrators of lung function decline and ultimate mortality in CF patients. Commonly found soil, water and plants, P. aeruginosa is considered an opportunist pathogen in that although it may infect healthy people, it rarely causes disease. However, more severe infections may occur in people who already have another illness. Once easily treated by several types of antibiotics, multiple-drug resistant strains known as Multidrug Resistant Pseudomonas aeruginosa (MDR-Pa) are becoming increasingly prevalent and much more difficult to treat. In the human body, P. aeruginosa forms large colonies over the surface of cells, known as biofilms, which help it avoid consumption (e.g., phagocytosis) by neutrophils in addition to other properties. A biofilm in general refers to a structured consortium of bacteria, embedded in a self-produced polymer matrix consisting of polysaccharide, protein and DNA.

Bacterial biofilms, in part, cause chronic infections because they show increased tolerance to antibiotics and resist phagocytosis, as well as other components of the innate and the adaptive immune system. As a consequence, a pronounced antibody response may develop, leading to immune complex-mediated chronic inflammation, dominated by polymorphonuclear leukocytes (e.g., neutrophils).

Chronic inflammation is a cause of the lung tissue damage in CF. Another contribution to persistence of chronic Pseudomonas (P.) aeruginosa lung infections in cystic fibrosis (CF) patients is due to biofilm-growing mucoid (alginate-producing) strains. One form of P. aeruginosa produces large amounts of a sugar (alginate) matrix and adheres to the damaged epithelial cell surfaces making the organism virtually impossible to eradicate. This type of P. aeruginosa is described as “mucoid”. In CF lungs, for example, the polysaccharide alginate is the major part of the P. aeruginosa biofilm matrix.

Biofilm growth in CF lungs is associated with an increased frequency of mutations, slow growth and adaptation of the bacteria to the conditions in the lungs, and resistant to antibiotic therapy. Low bacterial metabolic activity and increase of doubling times of the bacterial cells in CF lungs are responsible for some of the tolerance to antibiotics. Conversely, conventional resistance mechanisms, such as chromosomal β-lactamase, upregulated efflux pumps, and mutations of antibiotic target molecules in the bacteria, also contribute to the survival of P. aeruginosa biofilms. Biofilms can be prevented by early aggressive antibiotic prophylaxis or therapy, and they can be treated by chronic suppressive therapy. Hoiby, et al., Future Microbiol. 5 (11): 1663-74, 2010 [8].

P. aeruginosa is also particularly resistant to antimicrobial intervention because it has a large and diverse set of multidrug efflux pumps and an especially impermeable outer membrane (OM), and it can metabolize some antimicrobial compounds. In addition, P. aeruginosa is known to form biofilms that can further protect the bacteria. These biofilms are the subject of a significant body of research, and some investigators have proposed that clinical treatment of P. aeruginosa should involve elements that break down or prevent the formation of these biofilms (including but not limited to biofilm-like structures and microcolonies).

Another associated infection in CF patient is caused by Staphylococcus aureus bacteria, which causes pneumonia and skin infections. It is commonly found in the nose and on the surface of skin. Once easily treated by antibiotics, a methicillin-resistant strain known as MRSA is becoming more common.

Additional bacteria associated with CF respiratory infections include but are not limited to Burkholderia spp (B. multivorans and B. cinocepacia, etc.). This is a particularly aggressive bacterium that can cause a rapid decline in lung function and is often difficult to treat, as it may be resistant to most antibiotics.

Fungi may also contribute to lung impairment in CF patients such as Aspergillus fumigatus, a fungus or mold that is common in the environment. It may or may not cause symptoms if it is present in the lungs. However, some patients develop an allergy to the Aspergillus fumigatus. This allergy is called allergic bronchopulmonary aspergillosis or ABPA. Treatment of this organism depends on the symptoms.

The body mounts a strong immune response to fight bacteria and other organisms in individuals with CF. However, this constant, aggressive response to chronic infection may also lead to lung damage. In healthy lungs, white blood cells, or neutrophils, attack and eradicate bacteria. In CF patients, the neutrophils may function normally, but bacteria may not be eradicated. Further, when neutrophils engulf bacteria, they release chemicals that cause damage to the lungs. Even though the body can normally neutralize these chemicals, the ongoing presence of neutrophils in the airway overwhelms this process. Eventually, lung tissue is destroyed, airway gland secretion is increased, and cilia function decreases.

Bronchiectasis is one of the results of chronic inflammation, infection and mucus obstruction in CF lungs, where the muscular and elastic components of the airways are destroyed. This makes the airways weak and makes them dilated. The airways may balloon out to form a perfect hiding place for infection and pus. Tissue around the airways may also be damaged and scarred, blocking secretions from escaping the bronchial tree.

Medically related complications from producing thick and sticky mucus in the respiratory tract include but are not limited to: increased susceptibility to pneumonia, an increased susceptibility to sinus infections, an increased susceptibility to bronchial infections, an increased susceptibility to lung infections, induced adverse respiratory symptoms, etc., any one of which may lead to coughing, nasal congestion, etc. Additional complications may include induction of nasal polyps, increased sinus and nasal infections. Complications of altered secretions in other areas of the body include but are not limited to inflammation of the pancreas, and digestive tract disorders.

### D. Chronic Obstructive Pulmonary Disease (COPD).

COPD is considered the fifth leading cause of death worldwide (Pauwels & Rabe, 2004) and it will become, as predicted by the World Health Organization (WHO), the third leading cause of death worldwide by 2030 (worldwideweb.who.int/respiratory/copd/en/index.html). Even though airborne pollutants such as smoke from the burning fuel or coals can cause COPD, the main inducing factor is exposure to cigarette smoke.

COPD is a complex syndrome comprised of airway inflammation, mucociliary dysfunction and consequent airway structural destruction. This process is considered non-reversible. Upon the irritant challenge, the airway epithelial cells synthesize and release pro-inflammatory cytokines and chemokines such as IL-8, MIP-3alpha, which in turn recruit neutrophils, CD8+T-lymphocytes, B-Cells, macrophages and dendritic cells to the lumen of the airways. The matrix-metalloproteinases (MMP-6, MMP-9), among other mediators, cause airway injury and remodeling, eventually leading to airway obstruction.

### E. Respiratory Viruses.

Pathogenic viruses in some patients, and nuisance viruses in others, induce inflammation of the nasal and/or bronchial system. While in some patients the infection is self-limited with few additional symptoms, in other patients, including patients with pre-existing asthma, the inflammation persists leading to several types of conditions, including but not limited to asthma-like conditions, such as wheezing, where a patient has some asthma symptoms that resolve with short term treatment, asthma that progresses to a longer term condition that may or may not respond to treatment and when asthma causes severe symptoms, a patient may die.

Examples of viruses, e.g., respiratory viruses, that may contribute to induction of asthma attacks, include but are not limited to human Rhinovirus (HRV); flu (influenza) virus; parainfluenza virus; respiratory syncytial virus (RSV); metapneumovirus; coronavirus, and others. Busse, et al., “The Role of Viral Respiratory Infections in Asthma and Asthma Exacerbations.” Lancet, 4: 376 (9743): 826-834, 2010 [9].

Moreover, because factors, e.g., inflamed bronchial system; virus infection; allergens; bacteria; etc. may also induce or exuberate asthma, an allergic response to or in the presence of more than one factor, i.e., co-factors, e.g., an allergen such as pollen, pet dander, etc., in combination with inflammation induced by a viral invention, may lead to a severe asthma response including severe asthma attacks. Thus, a factor, such as an allergen, may serve as a co-factor with an inflammatory response to a viral infection, to worsen an asthma attack.

### F. Bacteria Exacerbators.

For one example, pneumococcus is a common cause of bacterial pneumonia, an illness that can be particularly serious in a person with asthma. In another example, other types of bacteria, such as Group A Streptococcus bacteria associated with strep throat infections, Group A Streptococcus bacteria, and Mycobacterium tuberculosis (TB) bacteria are associated with exacerbating asthma and respiratory infections in general.

### VIII. Skin Conditions and Diseases.

Compounds of the instant invention are useful to prevent or treat a plurality of dermatologic diseases or conditions, including but not limited to, dermatitis, atopic dermatitis, rash, pruritis, ankylosing spondylitis, eczema, acne, dandruff, cellulitis, psoriasis, rosacea, hives, shingles, lupus erythematosus, lichen planus, dermatitides, vasculitis, bullous diseases, and vitiligo.

Atopic dermatitis (AD) is a multifaceted, chronic relapsing inflammatory skin disease that is commonly associated with other atopic manifestations such as food allergy, allergic rhinitis, and asthma (N Engl J Med, 2008; 358 (14): 1483-1494 [10]; J Invest Dermatol, 2017; 137 (1): 26-30) [11]. Incidence of AD, also referred to as atopic eczema (the word “eczema” is a broad term that refers to various conditions causing inflammation of the skin), has increased 2- to 3-fold in industrialized nations since the 1970s, with approximately 15% to 20% of children and 1% to 3% of adults affected worldwide (Ann Nutr Metab. 2015; 66 (suppl 1): 8-16 [12]; J Am Acad Dermatol, 2014; 70 (2): 338-351 [13]). Population-based studies in the United States suggest that prevalence is about 10.7% for children and 7.2% for adults. Onset of disease commonly presents by 5 years of age, with the highest incidence occurring between the ages of 3 and 6 months, but it can occur at any age. Approximately 60% of patients develop disease in the first year of life and 90% within the first 5 years of life (J Am Acad Dermatol, 2014; 70 (2): 338-351 [13]). Twenty percent of children who develop AD before 2 years of age will have persisting symptoms of disease; 17% will have intermittent symptoms by 7 years of age. It is clear that AD is a chronic disease that is burdensome for many patients. A 2007 study further supports this claim, as an estimated 17.8 million persons, mostly undiagnosed, are living with AD in the United States (Dermatitis, 2007; 18 (2): 82-91 [14]).

AD disease burden often impacts overall quality of life and the social, academic, and occupational realms. The burden of AD is not limited to just the patient, because AD is a chronic relapsing skin disease that can persist into adulthood and burden of disease is frequently experienced by the patient's family. Direct costs include, but are not limited to, prescription and nonprescription costs, healthcare provider visits, hospital and emergency department visits, and hospitalizations. Indirect costs associated with AD include absenteeism from work, school, and physical activities; decreased productivity (presenteeism); and decreased quality of life (primarily due to sleep disturbance from itching, absenteeism, and time related to care) (Pediatr Dermatol, 2005; 22 (3): 192-199 [15]). A 2010 National Health Interview Study conducted in the United States estimated that 75% of people with eczema had visited a doctor at least once in the last year (J Invest Dermatol, 2015; 135 (1): 56-66 [16]).

AD is considered to be a chronic relapsing inflammatory skin condition. As a result, it generally presents in 3 different clinical phases: 1) acute AD (a vesicular, weeping, crusting eruption); 2) subacute AD (dry, scaly, erythematous papules and plaques); and 3) chronic AD (lichenification, thickening, from repeated scratching). Itch is the major symptom associated with impact on quality of life. Itch has been associated with mental distress and increased risk for suicidal ideation in those with AD (J Invest Dermatol, 2014; 134 (7): 1847-1854 [17]). Sleep disturbance is a frequent consequence of itch and is experienced by approximately two-thirds of patients with AD. Children with AD who experience sleep disturbances are associated with higher rates of developing attention-deficit/hyperactivity disorder, headaches, and short stature. Sleep disturbances experienced by adults with AD are associated with poor overall health perception (J Invest Dermatol, 2015; 135 (1): 56-66 [16]). Patients with atopic dermatitis are also more likely to develop ear infections (27% vs. 22%), streptococcal pharyngitis (8% vs. 3%), and urinary tract infections (8% vs. 3%).

Two major theories have been proposed to explain the cause of AD, the inside-out and outside-in hypotheses (Cutis, 2015; 96 (6): 359-361 [18]). The inside-out hypothesis proposes that allergic triggering leads to a weakened skin barrier that furthers allergen introduction and presentation. This would suggest that inflammation is the culprit for an impaired skin barrier, leading to increased penetration of allergens and microbes causing a reaction. The outside-in hypothesis proposes that the impaired skin barrier precedes AD and is required for immune dysregulation to occur. For example, the down-regulation of filaggrin (FLG), required for proper skin barrier function, could make the skin more susceptible to immune dysregulation and lead to AD. It is unlikely that the two theories are exclusive, and both most likely play a role in the pathogenesis of AD.

Certain genes associated with the immune system, for example cytokines that regulate immunoglobulin E (IgE) synthesis such as interleukin-4 (IL-4), interleukin-5 (IL-5), interleukin-12 (IL-12), interleukin-13 (IL-13), and granulocyte-macrophage colony-stimulating factor, have been associated with AD (N Engl J Med, 2008; 358 (14) [10]. Cytokines are mainly produced by type 1 and type 2 T helper lymphocytes (TH1 and TH2, respectively). TH1 cytokines (IL-12 and interferon-suppress IgE production, and TH2 cytokines (IL-5 and IL-13) increase IgE production. Patients with AD have a genetically determined dominance of TH2 cells that may decrease expression of FLG and other molecules found in the skin barrier.

The “atopic march” describes the tendency for AD to precede the development of other atopic diseases such as food allergies, asthma, and allergic rhinitis in a temporal sequence (Immunol Allergy Clin North Am, 2005; 25 (2): 231-246) [19]. Patients with atopic dermatitis, allergic rhinitis, and allergic asthma are considered to have the atopic triad. Approximately one-third of patients with AD develop asthma, and two-thirds develop allergic rhinitis (J Allergy Clin Immunol, 2007; 120 (3): 565-569 [20]; Immunol Allergy Clin North Am, 2010; 30 (3): 269-280) [21].

AD may be triggered by viral infections, food allergens, cosmetics, fragrance, weather, and other causes. Extremes of hot and cold weather are poorly tolerated by patients with AD and can lead to sweating and dry skin, respectively, initiating pruritus. Exposure to environmental allergens such as dust mites, pollens, molds, cigarette smoke, and dander from animals may exacerbate symptoms of AD (Cutis, 2016; 97 (5): 326-329) [22]. Food allergens may contribute to AD and the most commonly allergenic foods are eggs, milk, peanuts, wheat, soy, tree nuts, shellfish, and fish.

The goal of treatment in AD is to reduce symptoms, prevent exacerbations, treat superinfection, minimize treatment risks, and restore the integrity of the skin. In patients with mild disease, treatment goals may be achieved with topical therapies alone, unlike in patients with moderate to severe disease, whose management is challenging. Long-standing topical therapies are not sufficient for treating severe AD or to improve quality of life. Systemic immunomodulating agents are limited by side effects and by their effectiveness and biologics are costly and most applicable for disease that is resistant to other treatment modalities.

The American Academy of Dermatology has created simple diagnostic criteria based on symptoms and physical examination findings. Maintenance therapy consists of liberal use of emollients and daily bathing with soap-free cleansers. Use of topical corticosteroids is the first-line treatment for AD flare-ups. Pimecrolimus and tacrolimus are topical calcineurin inhibitors that can be used in conjunction with topical corticosteroids as first-line treatment. Ultraviolet phototherapy is a safe and effective treatment for moderate to severe AD when first-line treatments are not adequate. Anti-staphylococcal antibiotics are effective in treating secondary skin infections. Oral antihistamines are not recommended because they do not reduce pruritus. Evidence is lacking to support the use of integrative medicine in the treatment of atopic dermatitis. Newer medications approved by the U.S. Food and Drug Administration, such as crisaborole and dupilumab, can be effective in treating AD but are currently cost prohibitive for most patients.

Non-pharmacologic interventions such as the role of moisturizers and bathing practices help with treatment, maintenance, and prevention of flares. The application of moisturizers should be an integral part of the treatment of patients with AD. They are also components of maintenance therapy and prevention of flares. Bathing is suggested in patients with AD as part of treatment and maintenance; however, there is no standard for the frequency or duration of bathing appropriate for those with AD. Moisturizers should be applied soon after bathing to improve skin hydration. Limited use of non-soap cleansers (that are neutral to low pH, hypoallergenic, and fragrance-free) is recommended, and there are no data to support the use of bath water additives (oils, emollients, etc.). Wet-wrap therapy with or without topical corticosteroid can be recommended for patients with moderate to severe AD to decrease severity and water loss during flares. Topical corticosteroids (TCS) are used on both adults and pediatrics for the management of AD. A variety of factors must be considered to choose the proper TCS for treatment, such as patient preference and age. Based on patient risk factors and response, some monitoring may be required. Topical calcineurin inhibitors (TCI) can be used for the treatment of acute and chronic AD as well as maintenance therapy in both adults and children. They can serve as a steroid-sparing treatment; however, careful considerations must be made before prescribing. Patient education regarding TCI for AD is crucial since there are some adverse effects they may experience. Concomitant use of TCS and TCI can be used to treat AD. Topical antimicrobials and antiseptics such as bleach baths with intranasal mupirocin have been shown to be beneficial for AD patients; however, they are not routinely recommended. This therapy is mostly for patients with clinical signs of secondary bacterial infection to help reduce disease severity. Other forms of antimicrobial and antiseptic therapies have not been shown to be clinically helpful for AD. Topical antihistamines are not suggested for the treatment of AD due to the risk of absorption and contact dermatitis.

Phototherapy is typically used as a treatment for both acute and chronic AD in pediatric and adult patients. Phototherapy can be used as monotherapy or in combination with other topical therapies. However, caution must be taken due to drug interactions and increased risk of adverse effects. Phototherapy can be used in children; however, additional factors such as their psychological perspective may need to be considered when administering therapy.

Various factors must be considered when prescribing systemic agents, such as previous therapy failure or contraindications, as well as quality of life and disease severity. When using systemic agents, the minimal effective dose should be used, due to lack of optimal dosing, duration, or monitoring protocol guidance. Treatment is highly individualized and based on patient response, comorbidities, and history.

The following systemic therapies can be used off-label to treat AD. Some should only be considered as an alternative when other more commonly used off-label systemic therapies are not an option: cyclosporine, azathioprine, methotrexate, mycophenolate mofetil, and interferon gamma. Systemic steroids should be avoided, when possible, for the treatment of AD. They mainly serve for short-term bridge therapy to other systemic therapies or for acute severe exacerbations. There are limited data to support the use of oral antihistamines as a treatment of AD, they can be used to help with pruritus and some sedating antihistamines can help (short-term) with sleep loss due to AD.

### EXPERIMENTAL

The following examples serve to illustrate certain embodiments and aspects of the present invention and are not to be construed as limiting the scope thereof.

In the experimental disclosures which follow, the following abbreviations apply: N (normal); M (molar); mM (millimolar); gM (micromolar); mol (moles); mmol (millimoles); p.mol (micromoles); nmol (nanomoles); pmol (picomoles); g (grams); mg (milligrams); gg (micrograms); ng (nanograms); pg (picograms); L and (liters); ml (milliliters); gl (microliters); cm (centimeters); mm (millimeters); gm (micrometers); nm (nanometers); U (units); min (minute); s and sec (second); deg (degree); ° C. (degrees Centigrade/Celsius).

### Example I

### Synthesis

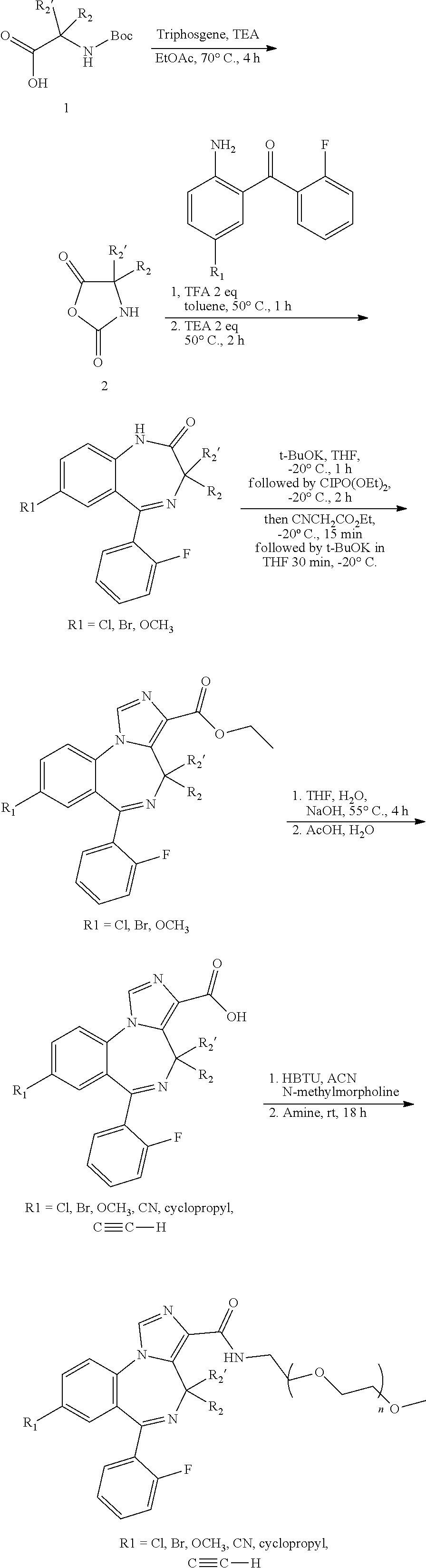

### Example II

### Synthesis of the Other R1 Groups

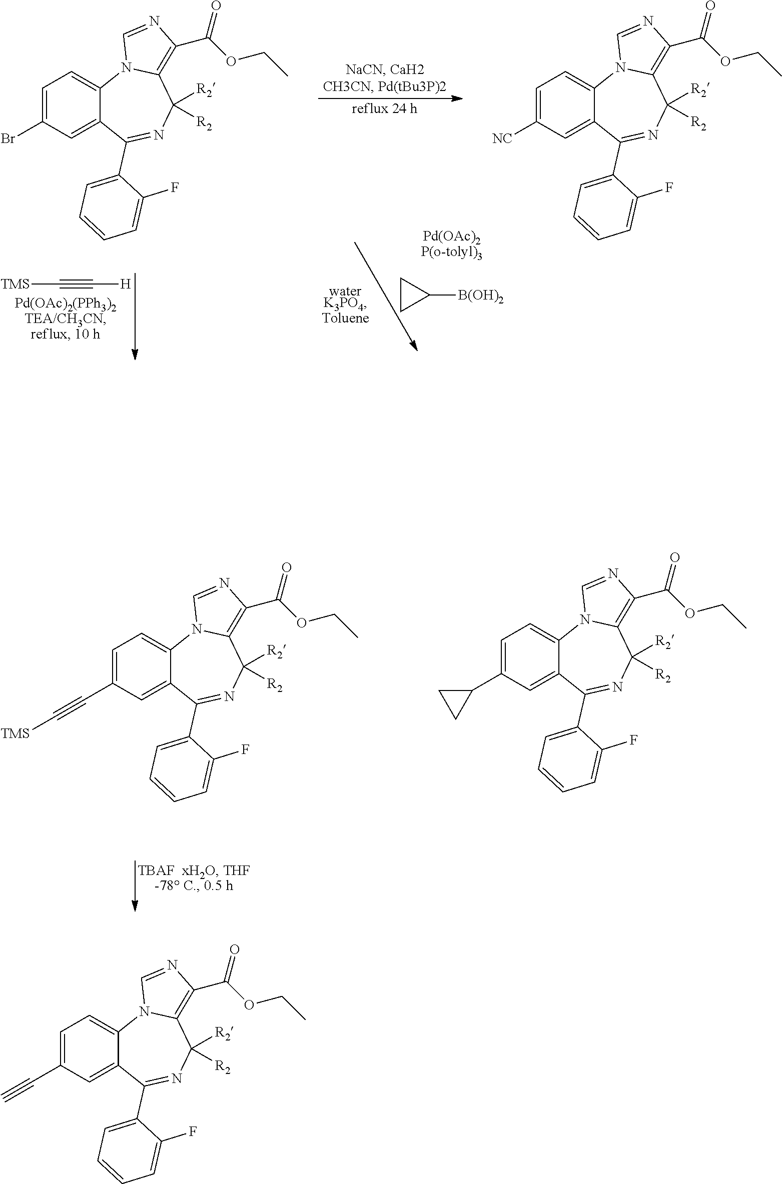

### Example III

### Synthesis of R2 and R2′ Sections

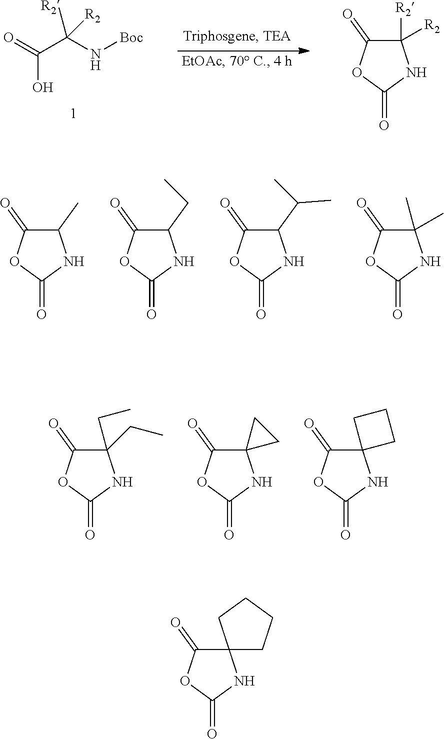

### Example IV

### Scheme 1. General Synthesis of Substituted 5-(2-fluorophenyl)-3-methyl-1,3-dihydro-2H-benzo[e][1,4]diazepin-2-ones

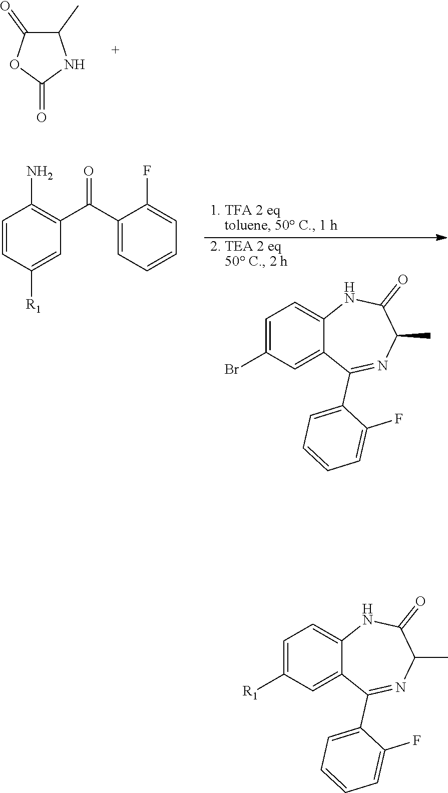

### Example V

### Preparation of (R)-7-bromo-5-(2-fluorophenyl)-3-methyl-1,3-dihydro-2H-benzo[e][1,4]diazepin-2-one

A mixture of 2-amino-5-bromo-2′-fluorobenzophenone (140.7 g, 478.4 mmol) and trifluoroacetic acid (73.3 mL, 956.7 mmol) in anhydrous toluene (2200 mL) was stirred at room temperature for 30 min to form a solution. N-carboxy-D-alanine anhydride (66.0 g, 574.0 mmol) was added and the reaction mixture was heated for 1 h at 50° C. After confirmation of >95% conversion 2-amino-5-bromo-2′-fluorobenzophenone (TLC, 50% ethyl acetate in hexanes, triethylamine (133.3 mL, 956.7 mmol) was added dropwise to the reaction mixture over 30 min, while maintaining the temperature at 50° C. After 2 h at 50° C., the intermediate TFA salt was converted (TLC, 50% ethyl acetate in hexanes). Upon cooling the reaction mixture to room temperature, solvents were removed under reduced pressure and the residue was dissolved in ethyl acetate (1500 mL) and water (1500 mL). The resulting biphasic mixture was separated and the organic layer was washed with 5% aqueous sodium bicarbonate solution (1500 mL) followed by 10% aqueous sodium chloride solution (1500 mL). The organic layer was dried over Na₂SO₄ and evaporated under reduced pressure. The residue was stripped with 10% ethyl acetate/heptane (25 mL×2) and slurried with 10% ethyl acetate in heptane (1700 mL) at 60° C. for 30 min to dissolve unreacted starting material 1. The reaction mixture was cooled to room temperature and stirred for an additional 2 h before the product was collected by filtration and washed with 10% ethyl acetate in heptane (50 mL×2) followed by heptane (50 mL×2). The solid was dried under vacuum at 40° C. to afford 5 as an off-white solid (127.0 g, 77.0%): ¹H NMR (500 MHZ, CDCl₃) δ 9.69 (s, 1H), 7.63-7.59 (m, 1H), 7.59 (dd, J=8.5, 2.3 Hz, 1H), 7.47 (dddd, J=8.2, 7.0, 5.0, 1.8 Hz, 1H), 7.36 (d, J=2.2 Hz, 1H), 7.26 (td, J=7.5, 1.1 Hz, 1H), 7.12 (d, J=8.6 Hz, 1H), 7.08 (ddd, J=10.2, 8.3, 1.1 Hz, 1H), 3.79 (q, J=6.5 Hz, 1H), 1.78 (d, J=6.5 Hz, 3H); ¹³C NMR (126 MHz, CDCl₃) δ 172.39 (s), 164.53 (s), 160.45 (d, ¹J_{CF}=251.9 Hz), 136.47 (s), 134.74 (s), 132.19 (d, ³J_{CF}=8.3 Hz), 132.03 (d, ¹J_{CF}=1.5 Hz, NOE coupling), 131.56 (d, ³J_{CF}=2.2 Hz), 130.15 (s), 127.13 (d, ²J_{CF}=12.4 Hz), 124.46 (d, ⁴J_{CF}=3.6 Hz), 122.98 (s), 116.54 (s), 116.29 (d, ²J_{CF}=21.5 Hz), 58.85 (s), 16.93 (s); ¹⁹F NMR (471 MHz, CDCl₃) δ −112.53; HRMS (ESI/IT-TOF): m/z [M+H]+ calcd for C₁₆H₁₃BrFN₂O: 347.0190; found: 347.0181; HPLC Purity: 99.2%; Optical Purity: 98.8% ee.

### Example VI

### Scheme 2. General Synthesis of Substituted Ethyl-6-(2-fluorophenyl)-4-methyl-4H-benzo[f]imidazo[1,5-a][1,4]diazepine-3-carboxylates

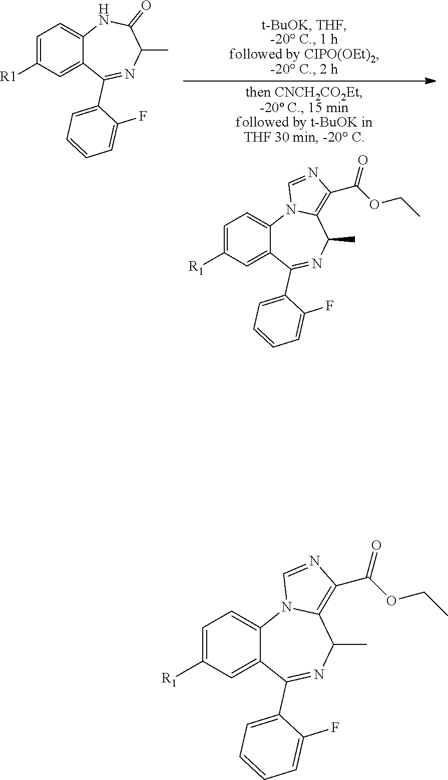

### Example VII

### Ethyl (R)-8-bromo-6-(2-fluorophenyl)-4-methyl-4H-benzo[f]imidazo[1,5-a][1,4]diazepine-3-carboxylate

(R)-7-bromo-5-(2-fluorophenyl)-3-methyl-1,3-dihydro-2H-benzo[e][1,4]diazepin-2-one (125.0 g, 360.0 mmol) in anhydrous tetrahydrofuran (2000 mL) was cooled to −20° C. using a dry ice/IPA bath. A solution of t-BuOK (52.5 g, 468.0 mmol) in tetrahydrofuran (300 mL) was added dropwise to the reaction mixture over 30 min, while maintaining a temperature of −20° C. Upon completion of the addition, the reaction mixture was allowed to stir for an additional 60 min at −20° C. Diethyl chlorophosphate (72.8 mL, 504.1 mmol) was then added dropwise to the reaction mixture over 15 min at −20° C. After 2 h at −20° C., the starting material was converted (TLC, 100% ethyl acetate). Ethyl isocyanoacetate (51.2 mL, 468.0 mmol) was added dropwise over 15 min while maintaining −20° C., followed by the dropwise addition of a solution of t-BuOK (52.5 g, 468.0 mmol) in tetrahydrofuran (300 mL) over 30 min at −20° C. Upon completion of the addition, the reaction mixture was allowed to warm to room temperature and stir for an additional 1 h, at which point the intermediate was fully converted (TLC, 100% ethyl acetate). The reaction mixture was diluted with 5% aqueous sodium bicarbonate (2000 mL) and ethyl acetate (2000 mL). The resulting emulsion was cleared by filtration and was separated after 30 min. The aqueous layer was extracted with ethyl acetate (2000 mL) and the combined organic layers were washed with 10% aqueous sodium bicarbonate solution (2000 mL) and 20% aqueous sodium chloride solution (2000 mL). The organic layer was dried over Na₂SO₄ and evaporated under reduced pressure. The residue was stripped with t-butyl methyl ether (200 mL×2) and slurried with t-butyl methyl ether (1000 mL) at 55° C. for 30 min. The mixture was stirred for 12 h at room temperature followed by filtration and washed with t-butyl methyl ether (100 mL×4). The solid was dried under vacuum at 40° C. to yield 7 as a white powder (96.6 g, 60.7%): ¹H NMR (500 MHZ, CDCl₃) δ 7.92 (s, 1H), 7.73 (dd, J=8.5, 2.2 Hz, 1H), 7.60 (dt, J=7.3, 3.9 Hz, 1H), 7.48 (d, J=8.6 Hz, 1H), 7.50-7.42 (m, 1H), 7.42 (d, J=2.2 Hz, 1H), 7.26 (td, J=7.5, 1.1 Hz, 1H), 7.10-7.02 (m, 1H), 6.71 (q, J=7.3 Hz, 1H), 4.54-4.28 (m, 2H), 1.42 (t, J=7.1 Hz, 3H), 1.29 (d, J=7.4 Hz, 3H); ¹³C NMR (126 MHz, CDCl₃) δ 162.93 (s), 162.67 (s), 160.10 (d, ¹J_{CF}=250.7 Hz), 141.59 (s), 134.85 (s), 134.75 (s), 133.68 (s), 133.05 (s), 132.12 (d, ³J_{CF}=8.2 Hz), 131.17 (s), 129.59 (s), 128.42 (d, ²J_{CF}=12.3 Hz), 124.57 (d, ⁴J_{CF}=3.3 Hz), 123.65 (s), 120.96 (s), 116.25 (d, ²J_{CF}=21.4 Hz), 60.82 (s), 50.12 (s), 14.87 (s), 14.43 (s); ¹⁹F NMR (471 MHz, CDCl₃) δ −112.36; HRMS (ESI/IT-TOF): m/z [M+H]+ calcd for C₂₁H₁₈BrFN₃O₂: 442.0561; found: 442.0563; HPLC Purity: 97.5%; Optical Purity: 99.0% ee.

### Example VIII

### Scheme 3. Synthesis of ethyl 8-cyano-6-(2-fluorophenyl)-4-methyl-4H-benzo[f]imidazo[1,5-a][1,4]diazepine-3-carboxylate

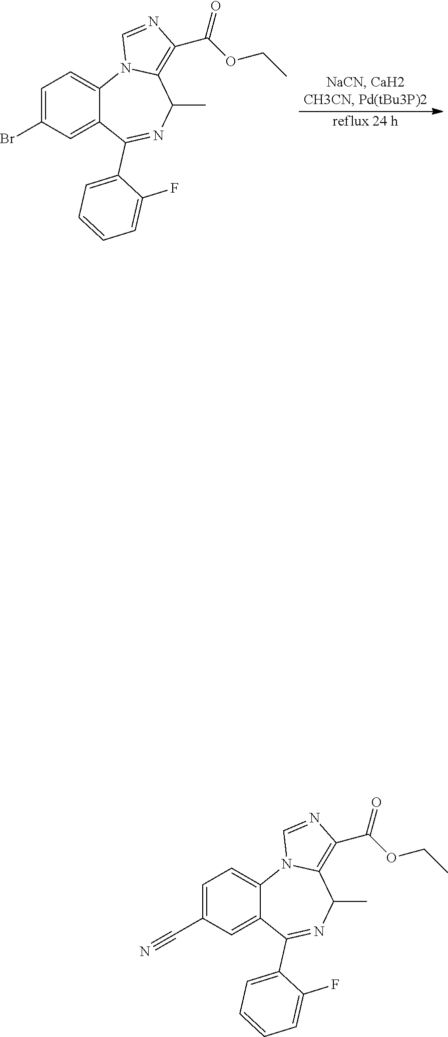 [SMILES: CCOC(=O)c1ncn2c1C(C)N=C(c1ccccc1F)c1cc(Br)ccc1-2 ; CCOC(=O)c1ncn2c1C(C)N=C(c1ccccc1F)c1cc(C#N)ccc1-2]

### Example IX

### Synthesis of ethyl 8-cyano-6-(2-fluorophenyl)-4-methyl-4H-benzo[f]imidazo[1,5-a][1,4]diazepine-3-carboxylate

A 20-mL screw-cap vial equipped with a magnetic stir bar was charged, 0.25 mmol ethyl 8-bromo-6-(2-fluorophenyl)-4-methyl-4H-benzo[f]imidazo[1,5-a][1,4]diazepine-3-carboxylate, acetonitrile (4 mL), finely ground NaCN (0.066 g; 1 mmol), and CaH₂ powder (10 mg; 0.24 mmol) and 15 mg of Pd (tBu₃P)₂. The reaction mixture was degassed under vacuum with argon; this process was repeated four times. The vial was sealed and vigorously stirred at 70° C. (oil bath) for 3 days. The reaction mixture was evaporated. The residue was treated with chloroform (5 mL) and the resultant suspension was filtered through a short Celite gel plug. 90 mg of the product was obtained (93% yield). ¹H NMR (500 MHz, CDCl₃) δ 7.97 (s, 1H), 7.87 (d, J=7.5 Hz, 1H), 7.72 (d, J=7.5 Hz, 1H), 7.65-7.59 (m, 2H), 7.48 (dq, J=7.5, 1.0 Hz, 1H), 7.28 (d, J=7.5, 1H), 7.04 (dt, J=7.5, 1.0 Hz, 1H), 6.74 (q, J=7.5 Hz, 0.78H), 4.45-4.31 (m, 2H), 2.15 (m, 0.26), 1.41 (t, J=7.0 Hz, 3H), 1.29 (dd, J=7.0, 3.5 Hz, 3H); ¹³C NMR (126 MHz, CDCl₃) δ 162.82, 162.30, 160.14 (d, 1¹J_{CF}=250.3 Hz), 141.85, 137.87, 135.01, 134.96, 134.64, 132.72 (d, ³J_{CF}=8.4 Hz), 131.35, 130.58, 130.27, 128.06 (d, ²J_{CF}=12.6 Hz), 124.96, 123.16, 117.10, 116.49 (d, ²J_{CF}=21.0 Hz), 111.76, 61.15, 50.29, 15.34, 14.55; ¹⁹F NMR (471 MHz, d6 DMSO) δ −112.12, Mass: C₂₂H₁₇FN₄O₂ m/z [M+H]+: 389.1; found: 389.1.

### Example X

### Scheme 4. Synthesis of ethyl (R)-8-cyclopropyl-6-(2-fluorophenyl)-4-methyl-4H-benzo[f]imidazo[1,5-a][1,4]diazepine-3-carboxylate

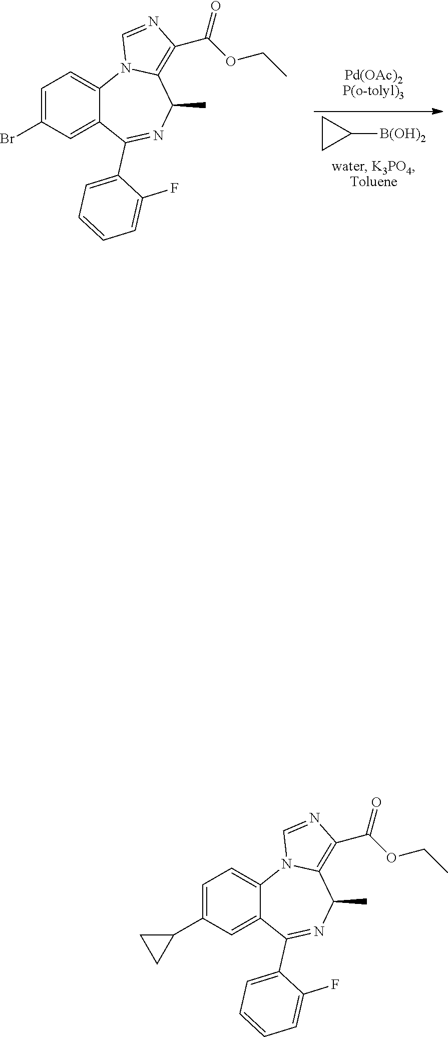

### Example XI

### Ethyl (R)-8-cyclopropyl-6-(2-fluorophenyl)-4-methyl-4H-benzo[f]imidazo[1,5-a][1,4]diazepine-3-carboxylate

To a solution of ethyl (R)-8-bromo-6-(2-fluorophenyl)-4-methyl-4H-benzo[f]imidazo[1,5-a][1,4]diazepine-3-carboxylate (2.1 g, 4.8 mmol) in toluene (20 mL) and water (1.4 mL), cyclopropyl boronic acid (1.0 g, 12.0 mmol), potassium phosphate (4.1 g, 19.2 mmol) and tri (O-tolyl) phosphine (85.5 mg, 0.28 mmol), Pd (OAc) 2 (31.5 mg, 0.14 mmol) were added under argon. A reflux condenser was attached and the mixture was degassed under vacuum with argon; this process was repeated four times. The mixture was stirred and heated to 100° C. After 12 h the reaction was completed on analysis by TLC (silica gel, DCM and 1% MeOH) and it was then cooled to rt. Water (10 mL) was added, and the mixture was extracted with EtOAc (3×15 mL), after which the filtrate was washed with brine (10 mL), dried (Na₂SO₄) and concentrated under reduced pressure. The black residue which resulted was purified by column chromatography using gradient of hexanes: 80% EtOAc and 20% chloroform to afford the desired 8-cyclopropyl-imidazodiazepine as white solid (1.26 g, 64.9% yield): ¹H NMR (500 MHz, CDCl₃) δ 7.88 (s, 1H), 7.55 (t, J=7.1 Hz, 1H), 7.44 (d, J=8.3 Hz, 1H), 7.42-7.32 (m, 1H), 7.19 (dd, J=12.9, 6.1 Hz, 2H), 6.98 (dd, J=18.9, 9.9 Hz, 2H), 6.64 (q, J=7.2 Hz, 1H), 4.55-4.14 (m, 2H), 1.92-1.73 (m, 1H), 1.38 (t, J=7.1 Hz, 3H), 1.24 (d, J=7.3 Hz, 3H), 1.03-0.87 (m, 2H), 0.68-0.48 (m, 2H); ¹³C NMR (126 MHZ, CDCl₃) δ 164.17, 163.12, 161.12, 159.12, 143.88, 141.58, 134.85, 132.09, 131.65, 131.58, 131.21, 129.22, 129.16, 129.04, 128.47, 127.87, 124.33, 121.95, 116.08, 115.91, 60.59, 50.05, 15.04, 14.64, 14.44, 9.91, 9.89; HRMS (ESI/IT-TOF) m/z: [M+H]⁺ Calcd for C₂₄H₂₃FN₃O₂ 404.1769; found 404.1763.

### Example XII

### Scheme 5. Synthesis of ethyl 8-ethynyl-6-(2-fluorophenyl)-4-methyl-4H-benzo[f]imidazo[1,5-a][1,4]diazepine-3-carboxylate

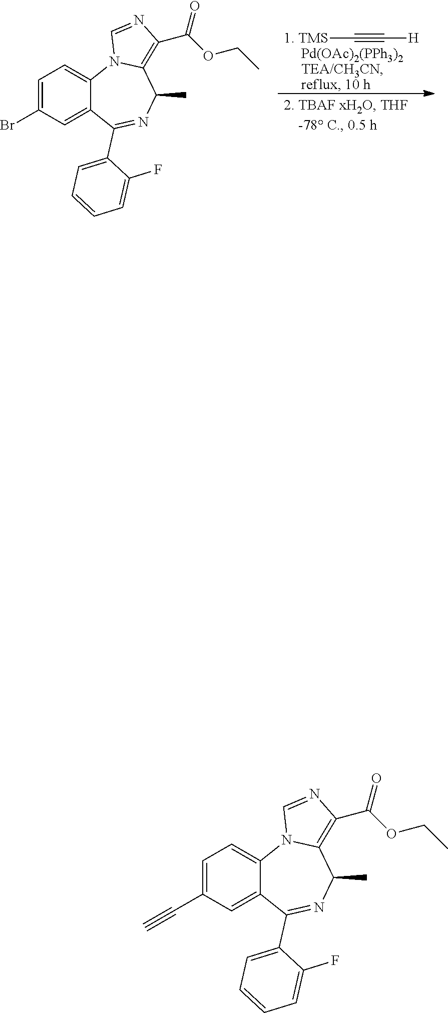

(R)-8-bromo-6-(2-fluorophenyl)-4-methyl-4H-benzo[f]imidazo[1,5-a][1,4]diazepine-3-carboxylate (55 g, 124.4 mmol) was dissolved in triethylamine (400 mL) and acetonitrile (550 mL). Trimethylsilylacetylene (18.3 g, 186.5 mmol) and bis(triphenylphosphine)-palladium (II) acetate (5.12 g, 6.8 mmol) were added. A reflux condenser was attached and the mixture was degassed under vacuum with argon; this process was repeated four times. The reaction mixture was heated to reflux under argon and stirred for 8 hours. The solution was cooled to rt, filtered through celite, and washed with EtOAc. The filtrate was concentrated under reduced pressure. The black residue which resulted was purified by a wash column (silica gel, EtOAc/hexanes 1:1) to afford the TMS-analog as an off-white solid (44.6 g, 77.7%): [α]²⁶_{D}=+28.2 (c 0.48, EtOAc); ¹H NMR (300 MHz, CDCl₃) δ 7.93 (s, 1H), 7.66 (d, J=7.9 Hz, 1H), 7.59 (t, J=7.3 Hz, 1H), 7.53 (d, J=8.3 Hz, 1H), 7.44 (td, J=7.3, 1.6 Hz, 1H), 7.36 (s, 1H), 7.24 (dd, J=10.9, 4.1 Hz, 1H), 7.04 (t, J=9.2 Hz, 1H), 6.68 (q, J=7.2 Hz, 1H), 4.53-4.23 (m, 2H), 1.41 (t, J=7.1 Hz, 3H), 1.25 (d, J=7.3 Hz, 3H), 0.23 (s, 9H); ¹³C NMR (75 MHZ, CDCl₃) δ 163.34 (s), 162.94 (s), 160.14 (d, J=250.6 Hz), 141.69 (s), 135.16 (s), 134.80 (s), 134.12-133.69 (m), 133.40 (s), 131.85 (d, J=8.3 Hz), 131.20 (s), 129.60 (s), 129.48 (s), 128.76 (d, J=13.2 Hz), 124.43 (d, J=3.4 Hz), 122.67 (s), 122.10 (s), 116.22 (d, J=21.5 Hz), 102.50 (s), 97.31 (s), 60.74 (s), 50.08 (s), 14.76 (s), 14.42 (s), −0.26 (s); HRMS (ESI/IT-TOF) m/z: [M+H] Calcd for C₂₆H₂₇FN₃O₂Si 460.1851; found 460.1833. The TMS analog (44.4 g, 96.6 mmol) was dissolved in THF (400 mL) and cooled to −78° C. This was treated with tetrabutylammonium fluoride hydrate (1 M solution in THF, 145 mmol), and this was followed by water (45 mL). The reaction mixture was stirred until the starting material was consumed as indicated by TLC (silica gel), about 1 h. The reaction mixture was allowed to warm to rt, and water (200 mL) was slowly added. The solution was extracted with EtOAc and the organic extracts were combined, washed with brine, dried (Na2SO4), and the solvent was removed under reduced pressure. The residue which resulted was purified by a wash column (silica gel, EtOAc/hexanes 1:1) to afford pure ethyl ester 1 as a white powder (36 g, 96.3%). [α]²⁶_{D}=+20.9 (c 0.89, EtOAc); ¹H NMR (300 MHz, CDCl₃) δ 7.93 (s, 1H), 7.69 (d, J=8.1 Hz, 1H), 7.57 (t, J=9.6 Hz, 2H), 7.48-7.36 (m, 2H), 7.24 (t, J=7.5 Hz, 1H), 7.02 (t, J=9.3 Hz, 1H), 6.69 (q, J=7.1 Hz, 1H), 4.54-4.27 (m, 2H), 3.15 (s, 1H), 1.40 (t, J=7.1 Hz, 3H), 1.27 (d, J=7.2 Hz, 3H). ¹³C NMR (75 MHz, CDCl₃) δ 163.20 (s), 162.91 (s), 160.08 (d, J=252.0 Hz), 141.64 (s), 135.17 (s), 134.86 (s), 134.49 (s), 133.88 (s), 131.93 (d, J=8.2 Hz), 131.17 (s), 129.59 (s), 129.50 (s), 128.64 (d, J=12.6 Hz), 124.47 (d, J=3.3 Hz), 122.23 (s), 121.62 (s), 116.16 (d, J=21.5 Hz), 81.40 (s), 79.78 (s), 60.75 (s), 50.07 (s), 14.85 (s), 14.41 (s); HRMS (ESI/IT-TOF) m/z: [M+H]+ Calcd for C₂₃H₁₉FN₃O₂ 388.1456; found 388.1439.

### Example XIII

### Scheme 6. General Synthesis of Substituted 6-(2-fluorophenyl)-4-methyl-4H-benzo[f]imidazo[1,5-a][1,4]diazepine-3-carboxylic acids

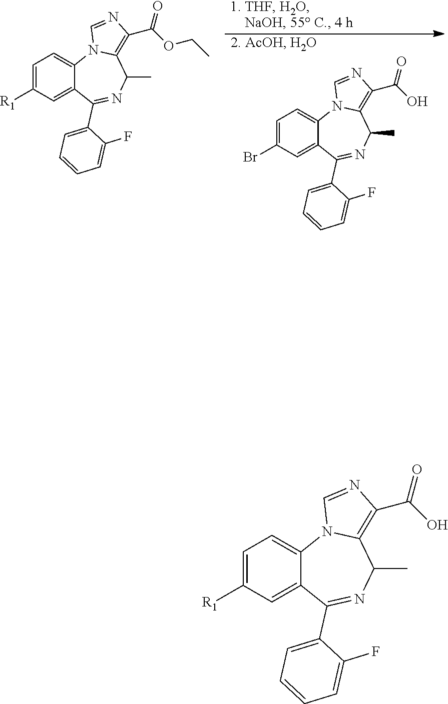

### Example XIV

### (R)-8-bromo-6-(2-fluorophenyl)-4-methyl-4H-benzo[f]imidazo[1,5-a][1,4]diazepine-3-carboxylic acid

To a solution of ethyl (R)-8-bromo-6-(2-fluorophenyl)-4-methyl-4H-benzo[f]imidazo[1,5-a][1,4]diazepine-3-carboxylate (96.0 g, 217.0 mmol) in tetrahydrofuran (1500 mL) was added 500 mL of a 1.74M aqueous solution of sodium hydroxide dropwise over 15 min while keeping the temperature at 30° C. The reaction mixture was heated for 4 h at 55° C. to convert the staring material (TLC, 100% ethyl acetate). The reaction mixture was then cooled to room temperature followed by the addition of water (1000 mL) and acetic acid (74.5 mL, 1302.3 mmol) dropwise over 15 min while maintaining the temperature at 25° C. Tetrahydrofuran was removed under reduced pressure and methanol (750 mL) was added to the mixture and heated for 30 min at 60° C. After cooling to room temperature, the mixture was stirring for 12 h. The solid was filtered and washed with water (200 mL×4). The solid was dried under vacuum at 40° C. to yield MIDD0301 as an off-white powder (87.7 g, 97.5%). The purity was 97.2% by HPLC. Recrystallization in ethanol gave 93.8% yield with a 98.8% purity. The optical purity: >99.0% ee. ¹H NMR (500 MHZ, D₆-DMSO) δ 12.83 (s, 1H), 8.42 (s, 1H), 8.09-7.92 (m, 1H), 7.89 (d, J=8.7 Hz, 1H), 7.59 (t, J=5.5 Hz, 1H), 7.55 (dtd, J=7.5, 5.4, 2.5 Hz, 1H), 7.36-7.30 (m, 2H), 7.23 (dd, J=10.7, 8.2 Hz, 1H), 6.52 (q, J=7.3 Hz, 1H), 1.17 (d, J=7.3 Hz, 3H); ¹³C NMR (126 MHZ, D6-DMSO) δ 164.68 (s), 162.37 (s), 159.85 (d, ¹J_{CF}=248.3 Hz), 140.86 (s), 136.64 (s), 135.49 (s), 134.02 (s), 132.74 (d, ³J_{CF}=7.9 Hz), 132.38 (s), 131.93 (s), 130.89 (s), 129.58 (s), 128.70 (d, ²J_{CF}=11.7 Hz), 125.64 (s), 125.18 (d, ⁴J_{CF}=2.8 Hz), 120.26 (s), 116.43 (d, ²J_{CF}=21.1 Hz), 49.83 (s), 15.05 (s); ¹⁹F NMR (471 MHZ, DMSO) δ −114.15; HRMS (ESI/IT-TOF): m/z [M+H]+ calcd for C₁₉H₁₄BrFN₃O₂: 414.0248; found: 414.0246.

### Example XV

### Scheme 7. General Synthesis of Substituted 6-(2-fluorophenyl)-4-methyl-4H-benzo[f]imidazo[1,5-a][1,4]diazepine-3-carboxylic acids

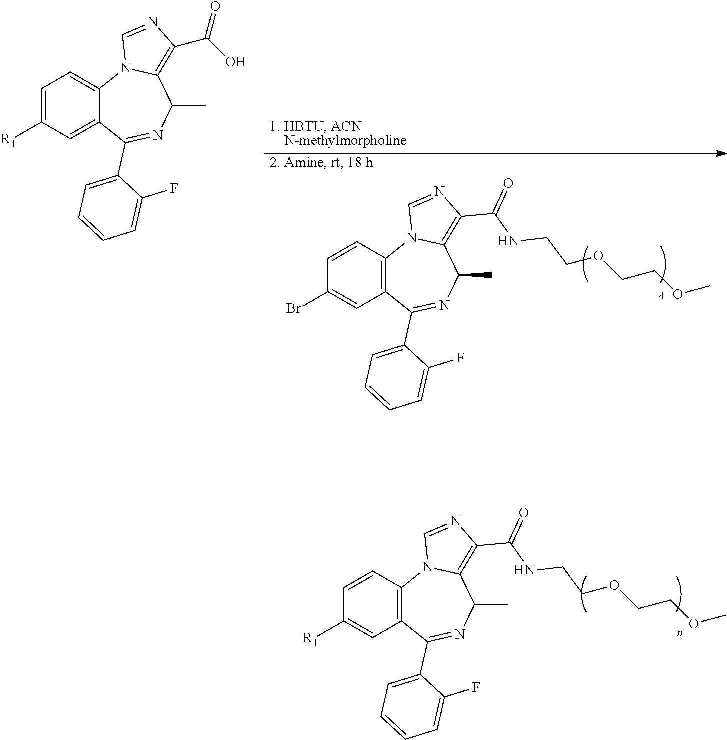

### Example XVI

### (R)-8-bromo-6-(2-fluorophenyl)-N-(2,5,8,11,14-pentaoxahexadecan-16-yl)-4-methyl-4H-benzo[f]imidazo[1,5-a][1,4]diazepine-3-carboxamide PI320

(R)-8-bromo-6-(2-fluorophenyl)-4-methyl-4H-benzo[f]imidazo[1,5-a][1,4]diazepine-3-carboxylic acid (0.5 mmol, 207 mg), HBTU (280 mg, 0.52 mmol) and N-methylmorpholine (0.202 mL, 2.0 mmol) were stirred in dry acetonitrile (5 mL), under nitrogen at RT for 10 minutes. Solution almost becomes clear. Amine (125 mg or 125 uL) was added and stirring was continued for 18 hours. After the completion of the reaction (TLC, silica gel) the solvent was removed under reduced pressure. The residue was dissolved in CHCl3 and treated with aq NH4Cl and brine followed by MgSO4 dry. The residue was purified by column chromatography using a gradient of 0-10% methanol in CHCl3 (30 CV). Yield 62% (201 mg). ¹H NMR (500 MHz, d6 DMSO) δ 8.42 (s, 1H), 8.06 (t, J=7.5 Hz, 1H), 7.93 (d, J=7.5, 1H), 7.88 (d, J=7.5, 1H), 7.58 (t, J=7.5, 1H), 7.54 (q, J=7.5, 1H), 7.32 (t, J=7.5, 1H), 7.21 (t, J=7.5, 1H), 6.66 (q, J=7.5 Hz, 0.69H), 3.53-3.46 (m, 16H), 3.41-3.38 (m, 2H), 3.328 (NH, 1H), 3.21 (s, 3H), 1.99 (m, 0.26) H, 1.16 (d, J=7.0 Hz, 3H); ¹³C NMR (126 MHz, d₆ DMSO) δ 162.28, 161.75, 159.34 (d, 1¹J_{CF}=248.2 Hz), 137.56, 135.34, 135.0 (d, ³J_{CF}=8.2 Hz), 133.60, 132.18, 131.93, 131.43, 130.63, 130.41, 128.37 (d, ²J_{CF}=12.6 Hz), 125.08 11 (d, ⁴J_{CF}=3.8 Hz), 124.69, 119.66, 115.89 (d, ²J_{CF}=21.2 Hz), 71.25, 69.79, 69.76, 69.75, 69.74, 69.73, 69.73, 69.72, 69.54, 68.96, 58.01, 49.01, 14.72. ¹⁹F NMR (471 MHz, d6 DMSO) δ −109.48. Mass: m/z [M+H]: C₃₀H₃₆BrFN₄O₆ 647.1 found: 647.1.

### Example XVII

### Inflammatory Bowel Disease Model

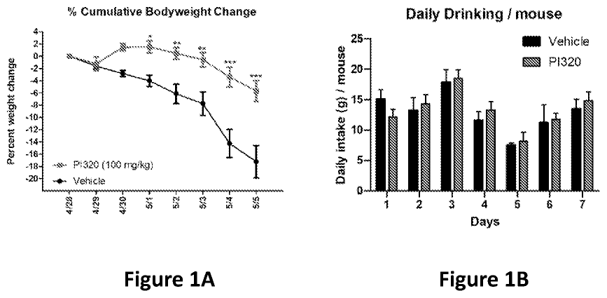

Ten (10) week old female Swiss Webster mice were offered 3% Dextran sodium sulfate (DSS) water to induce ulcerative colitis. Group 1 (n=8) received daily peanut butter (200 mg) and group 2 (n=8) received 100 mg/kg PI320 in peanut butter. The animals were weighted daily. See, FIGS. 1A and 1B.

Both groups of groups lost weight during the first day (FIG. 1A). PI320 treated animals regained weight on day 2 sustained their weight for days. A small drop in weight in comparison to the initial weight was observed on days 6 and 7. In contrast, vehicle treated animals started to lose weight throughout the study reaching the endpoint of 15% weight loss on day 7. The consumption of 3% DSS drinking water was the same for both groups (FIG. 1B).

MPO assay: Whole colons were homogenized in phosphate buffer and centrifuge at 10,000 rpm at 4° C. from 10 min. The protein concentration supernatant was determined with a BCA assay and adjusted to 200 μg/ml with the addition of phosphate buffer. A daily standard solution for the hydrogen peroxide solution was prepared to a concentration of 0.0024% from a 35% v/v stock. The phosphate buffer that was prepared for the BCA assay was utilized for the dilution step of this assay. O-dianisidine dihydrogen chloride was prepared at a concentration of 167 μg/mL in phosphate buffer and sonicated for 30 min to dissolve in the buffer. 8 L of protein solution was transferred to a 384 well plate containing 62 μL of the o-dianisidine solution. The reaction is initiated with the addition of 10 μL of the hydrogen peroxide solution prepared above. The conversion of the o-dianisidine was mitigated by a secondary oxidation after the hydrogen peroxide is converted into hypochlorite. The product of this reaction shifts the absorbance to the λₘₐₓ to 460 nm. The plate is then measured over a 15 min period in the plate reader in 30 sec intervals. Four replicates on each homogenate sample were conducted for each of the MPO assays.

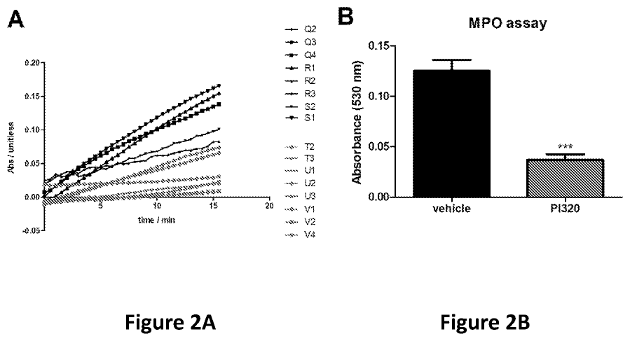

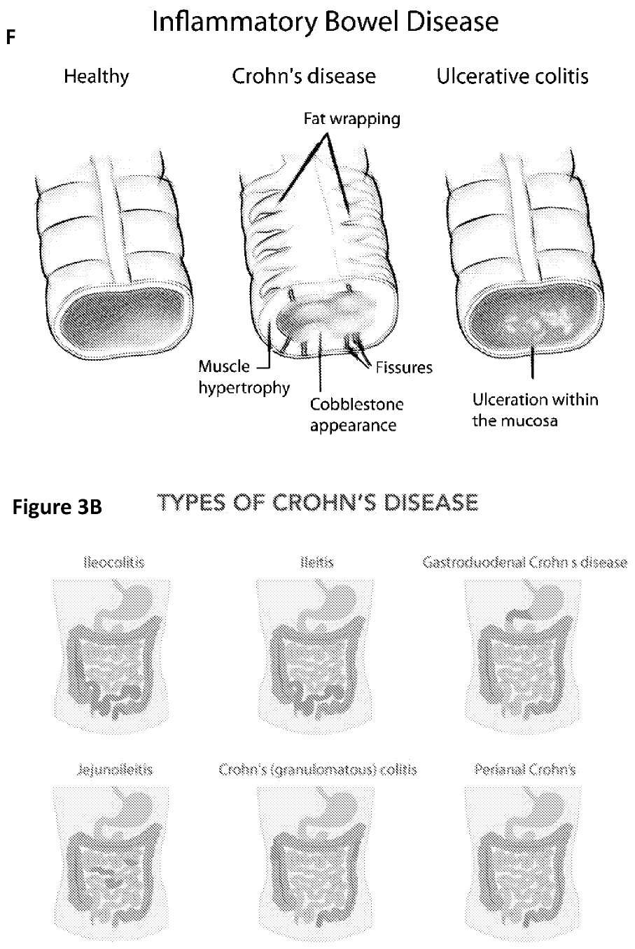

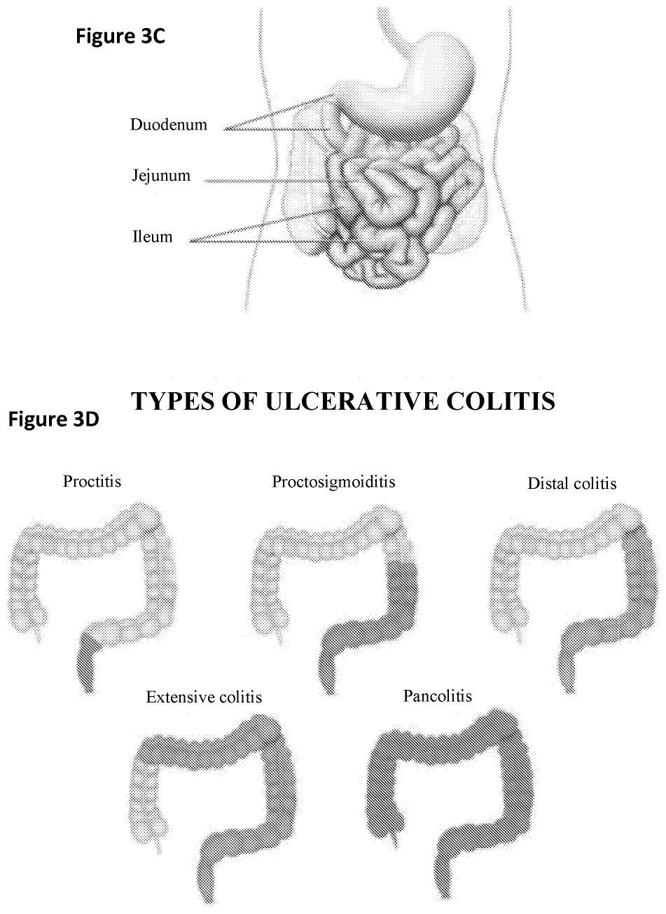

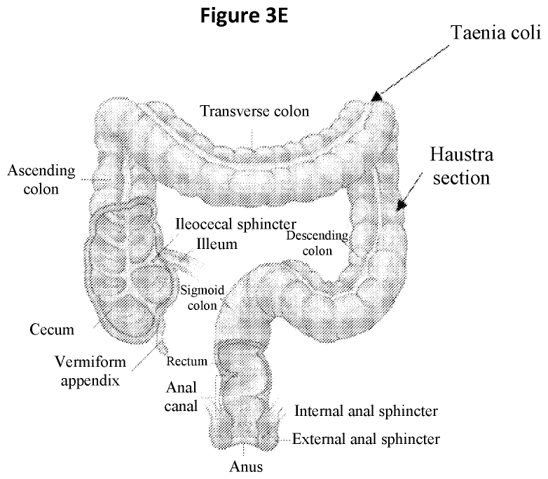

A significant difference MPO levels were observed. The conversion of MPO substrate was significantly higher for all PI320 treated animals (FIG. 2A). Myeloperoxidase, an enzyme expressed by neuropils and is upregulated in the colon of the vehicle group but greatly reduced in the PI320 treated mice. The amount of oxidized o-dianisidine was significantly different after a 15-minute incubation time (FIG. 2A).

### Example XVIII

### Use of PI320 in Steroid Resistant Mouse Asthma Model

Female A/J mice are purchased from Jackson Laboratory (Bar Harbor, ME) and female Swiss Webster mice are purchased from Charles River Laboratory (Wilmington, MA). Animals are given ad libitum access to food and water. Test drugs are prepared for nebulization as follows: a 3.0 mg/ml solution of albuterol (Millipore Sigma) is prepared in phosphate buffered saline (137 mM NaCl, 2.7 mM KCl, 10 mM Na2HPO4, and 1.8 mM KH2PO4) and PI320 is prepared as 3.2 mg/ml solution in water with 0.17% Tween-20. Airway hyperresponsiveness measurements, sRaw, are obtained using a Buxco FinePointe Non-Invasive Airway Mechanics instrument (DSI, St. Paul, MN) as described previously (ACS Pharmacol Transl Sci 3, 1381-1390) [23].

Efficacy of control drug (albuterol) and PI320 are determined by reduction of sRAW in mice compared to vehicle treated control mice. Here, mouse lung inflammation is induced with INFγ and bacterial lipopolysaccharide (LPS) via intratracheal installation. Elevated levels of IFNγ in the lung are observed for neutrophilic asthma patients, which respond poorly to steroid treatment (Eur Respir J 11, 312-316) [24]. One contributing factor is the impaired nuclear translocation of the liganded glucocorticoid receptor in pulmonary macrophages (Am J Respir Crit Care Med 191, 54-62) [25]. LPS simulates a microbial infection leading to macrophage-mediated inflammatory response (J Immunol 185, 4401-4409) [26].

The tracheal installation of INFγ/LPS increases airway hyper-responsiveness to an inhaled challenge agent as measured by sRAW, which is based on the breathing volume and frequency. The challenge agent, methacholine, is administered three times over 24 minutes resulting in sRAW of approximately 8.0 cmH₂O*sec. Mice that receive nebulized albuterol or PI320 (each at 7.2 mg/kg) demonstrate statistically significant reductions (p≤0.005 determined by 2-way ANOVA compared to vehicle control) of AHR when administered prior to second and third methacholine application. In contrast, aerosolized PI301 is not efficacious in this model at 3 mg/kg (ACS Pharmacol Transl Sci 3, 1381-1390) [23].

Drug efficacy can also be demonstrated using A/J mice that display pronounced airway hyper-responsiveness to methacholine without INFγ/LPS or allergen pretreatment due their unique genetic profile (Proc Natl Acad Sci USA 108, 12787-12792) [27].

Results show that PI320 is at least as effective as albuterol in reducing methacholine induced airway hyper-responsiveness when administered via inhalation. PI320 thus provides an effective treatment in patients who do not tolerate albuterol (a beta-adrenergic receptor agonist) or whose disease has become resistant to albuterol effects.

### Example XIX

### Use of PI320 in MC903 Mouse AD Model

An established mouse model of AD is used to evaluate the efficacy of PI320 in reducing ear thickness following MC903 challenge (for background on the model see: J Invest Dermatol 138, 2606-2616 [28]; Proc Natl Acad Sci USA, 103, 11736-11741 [29]; Sci Transl Med, 5, 170ra116 [30]; J Invest Dermatol, 138, 1555-1563 [31]). Ear thickness is used as a measure of inflammation caused by the chemical irritant.

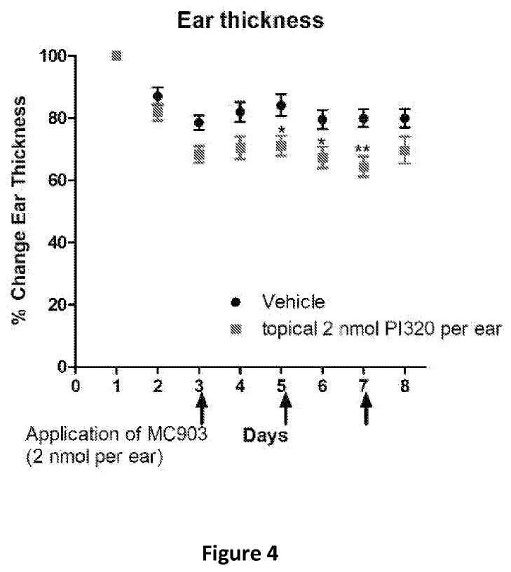

Briefly, commercially sourced female Swiss Webster mice (Charles River Laboratory, Wilmington, MA) at approximately seven weeks of age are used. Sixteen mice are housed under 7:00 AM-7:00 PM 12 h light cycle and administered 2 nmol MC903 on each ear on Monday, Wednesday and Friday for two weeks. The ear thickness increased from 0.3 mm to 0.7 mm during that time. During the third week, a group of eight mice were administered 2 nmol of PI320 topically on both ears (10 ul of a 100 uM solution in ethanol on each side of both ears). The control group received vehicle ethanol. Ear thickness is measured using calipers. Data show that topical PI320 significantly reduces ear thickness (*=p≤0.05; **p≤0.01) compared to vehicle control. See, FIG. 4.

The increase of ear thickness is based on the increased diameter of stratum, epidermis, and dermis as determined by histological processing and analysis using standard methods. The thickening of the stratum is based on parakeratosis and hyperkeratosis, the thickening of the epidermis is based on acanthosis and spongiosis and the thickening of the dermis is based on inflammatory infiltration. This reflects increased numbers of neutrophils, mast cells, and, to a lesser extent, eosinophils.

### Example XX

### GABA_{A} receptor binding

Rat brain membranes are prepared from frozen tissue that was thawed on ice, homogenized on ice in 10 volumes of cold lysis buffer (50 mM Tris HCl, pH 7.4, containing protease inhibitor cocktail from Roche) using a Polytron homogenizer (6 pulses and 10 seconds per pulse). The homogenate is centrifuged at 1,000×g for 10 min at 4° C. to obtain supernatant. The supernatant is then centrifuged at 40,000×g for 20 min, and then the resulting supernatant is decanted and replaced with the same ice-cold lysis buffer. Two or three additional rounds of homogenization-centrifugation are performed to ensure thorough homogenization and also to wash out endogenous ligands. The final pellet is resuspended in the same buffer and homogenized one last time. The rat brain suspension is diluted in buffer (50 mM Tris HCl, 2.5 mM CaCl₂), pH 7.4), followed by the addition of [3H]-flunitrazepam (0.6-4.0 nM in DMSO) and PI320 or PI310 in DMSO at different concentrations to reach a final of volume of 125 ul per well. GABA_{A}R subtypes that bind flunitrazepam consist of α_{1-3,5,6}β₁₋₃γ₁₋₃/δ GABA_{A}Rs. [32-35] Relative expression of GABA_{A}Rs in the brain consist of 43% α₁β₂γ₂, 15% α₂β₃γ₂+8% α₂βγ₁, 10% α₃β₃γ₂, 6% α₄βγ/δ, 4% α₅β₃γ₂, and 4% α₆β₂γ₂/δ. [36] Thus, compounds such as clonazepam with high activity in this assay bind predominately α₁₋₃β₂₋₃γ₁₋₂ GABA_{A}Rs.

Total binding and nonspecific binding are determined with reference compound clonazepam. In brief, plates are usually incubated at room temperature and in the dark for 90 min. Reactions are stopped by vacuum filtration onto 0.3% polyethyleneimine (PEI) soaked 96-well filter mats using a 96-well Filtermate harvester, followed by three washes with cold PBS buffer. Scintillation cocktail is then melted onto the microwave-dried filters on a hot plate and radioactivity counted in a Microbeta counter. The data (n=6) are analyzed by nonlinear regression.

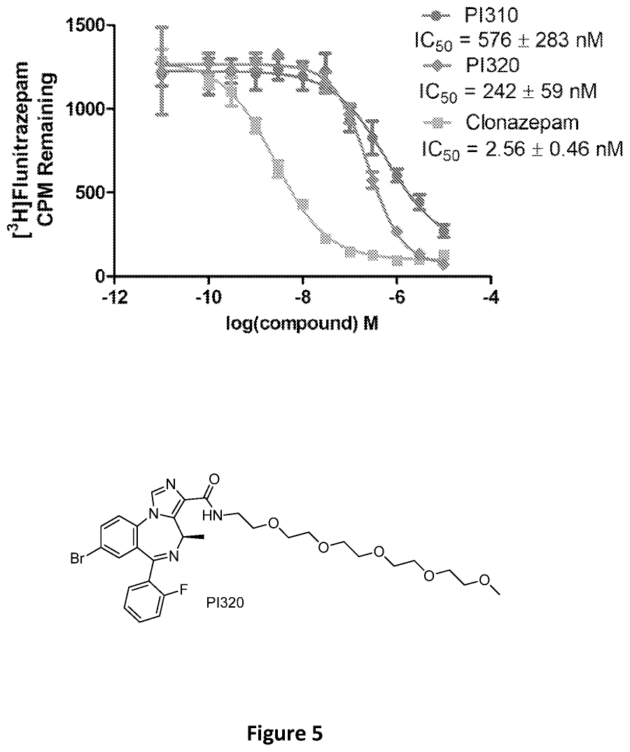

FIG. 5 shows exemplary binding of PI310 and PI320 toward the GABA (A) receptors.

An IC50 of 576 nM was observed for PI310, which is significantly lower than the reported IC50 of 72 nM for MIDD0301. [37] Thus the conjugation to 6-(4-phenylbutoxy) hexan-1-amine reduced binding eight fold. The GABA_{A}R affinity of PI320 is significantly stronger with an IC50 of 242 nM. Overall, this confirms that even large substituents are tolerated in this position for imidazobenzodiazepines, however, the hydrophobic nature of the substituents has an influence on binding affinity.

### Example XXI

### Molecular Docking

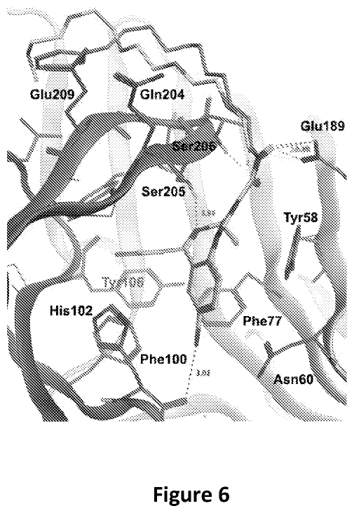

Molecular docking was performed with MOE 2015.1001 (Molecular Operating Environment, Montreal, Canada). The 6HUO [2] protein database file was downloaded via worldwideweb.ncbi.nlm.nih.gov and prepared for docking using the available MOE feature. The pharmacophore query editor was used determine location and annotations for alprazolam bound to the α₁β₃γ_{2L} GABA_{A} receptor. Docking was performed with both thermodynamically rotamers (Mol Pharm, 2020, 17:1182-1192 [38]) of PI310 and PI320 using the pharmacophore of alprazolam for placement and affinity dG for scoring. Refinement was determined with a rigid receptor structure. See, FIG. 6 shows exemplary docking study with PI310 and PI320. Overlay of docked conformations of PI310 (magenta) and PI320 (green) in the complex with the α₁β₃γ_{2L} GAB_{A}A receptor using structure 6HUO (Nature, 2019, 565:454-459 [2]). The α₁/γ₂ interface is indicated as α₁ (red) and γ₂ (cyan). Hydrogen and halogen bonds are indicated as dashed lines.

The exemplary ligands are docked at the alprazolam binding site, which is located at the α1⁺/γ2⁺ interface. The bromine atom is near Asn60 and His102. Experiments with isocyanate substituted diazepam have resulted in alkylation of these residues (ACS Chem Biol, 2014, 9:1846-1853 [39]). Halogen bonding with Phe100 located on al subunit is possible. Hydrogen bond interactions have been reported for other imidazodiazepines such as Hz166 with Ser205 (ARKIVOC 2020, 242-256 [40]). Amides PI310 and PI320 can additionally interact with Glu189 located on γ2 subunit. The alkyl chain of both compounds is located on the periphery of the protein complex and mostly exposed to water and hydrophilic amino acid residues. In contrast to the 6-(4-phenylbutoxy) hexyl side chain, the 2,5,8,11,14-pentaoxahexadecanyl side chain has multiple hydrogen accepting groups that can interact form hydrogen bond interaction with charged residues of the protein surface resulting in a higher affinity for GABA_{A}Rs.

### Example XXII

### Synthesis of Compounds PI350-PI352

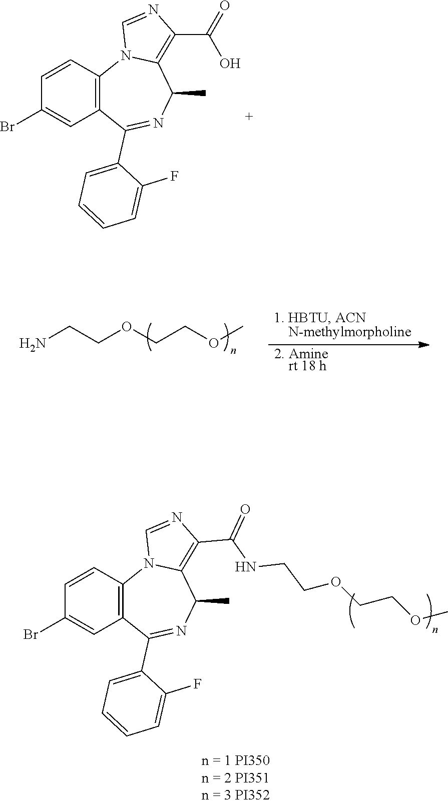

Racemic MIDD0301 acid (1 mmol, 416 mg), HATU (360 mg, 1.05 mmol) and N-methylmorpholine (0.404 mL, 4.0 mmol) were stirred in dry acetonitrile (10 mL), under nitrogen at RT for 10 minutes. Solution almost becomes clear. Amine (120 mg or 120 uL) was added and stirring was continued for 18 hours. After the completion of the reaction (TLC, silica gel) the solvent was removed under reduced pressure. The residue was dissolved in CHCl3 and treated with aq NH4Cl and brine followed by MgSO4 dry. The residue was loaded on a precolumn with CHCL3:Methanol 9:1 and separated by Biotage: 0-10% methanol in CHCl3 (25 CV).

PI350: Yield 91% 468 mg, ¹H NMR (500 MHz, CDCl₃) δ 7.91 (s, 1H), 7.67 (d, J=7.5 Hz, 1H), 7.60 (t, J=7.4 Hz, 1H), 7.52 (s, NH) 7.44-7.37 (m, 3H), 7.22 (dt, J=6.0, 1.0 Hz, 1H), 6.99 (t, J=9.0 Hz, 0.76H), 6.86 (q, J=7.2 Hz, 1H), 4.29 (m, 0.24H), 3.66-3.51 (m, 8H), 3.37 (s, 3H), 2.21 (m, 0.47H), 1.25 (d, J=7.3 Hz, 3H). ¹³C NMR (126 MHz, CDCl₃) δ 162.81, 162.35, 160.18 (d, J=250.8 Hz), 138.94, 134.72, 133.95, 133.50, 133.06, 132.04 (d, J=8.3 Hz), 131.83, 131.43, 131.38, 128.69 (d, J=12.3 Hz), 124.60 (d, J=3.2 Hz), 123.61, 120.77, 116.20 (d, J=21.5 Hz), 71.97, 70.40, 70.29, 59.17, 50.02, 38.74, 15.13. ¹⁹F NMR (471 MHz, CDCl₃) δ −112.06 (minor rotamer), −112.33 (major rotamer).

PI351: Yield 88% 491 mg. ¹H NMR (500 MHZ, CDCl₃) δ 7.79 (s, 1H), 7.67 (d, J=7.5 Hz, 1H), 7.60 (t, J=7.4 Hz, 1H), 7.50 (s, NH) 7.44-7.37 (m, 3H), 7.22 (dt, J=6.0, 1.0 Hz, 1H), 7.00 (t, J=9.0 Hz, 0.74H), 6.86 (q, J=7.2 Hz, 1H), 4.29 (m, 0.26H), 3.66-3.51 (m, 12H), 3.37 (s, 3H), 2.06 (m, 0.47H), 1.25 (d, J=7.3 Hz, 3H). ¹³C NMR (126 MHz, CDCl₃) δ 162.79, 162.34, 160.18 (d, J=250.8 Hz), 138.94, 134.72, 133.94, 133.50, 133.44, 133.06, 132.04 (d, J=8.3 Hz), 131.83, 131.44, 131.38, 128.69 (d, J=12.3 Hz), 124.61 (d, J=3.2 Hz), 123.61, 120.77, 116.19 (d, J=21.5 Hz), 72.04, 70.66, 70.60, 70.51, 70.21, 59.13, 50.00, 38.76, 15.10. ¹⁹F NMR (471 MHz, CDCl₃) δ −112.07 (minor rotamer), −112.33 (major rotamer).

PI352: Yield 98% 591 mg, ¹H NMR (500 MHz, CDCl₃) δ 7.79 (s, 1H), 7.67 (d, J=7.5 Hz, 1H), 7.59 (t, J=7.4 Hz, 1H), 7.51 (s, NH) 7.41-7.35 (m, 3H), 7.21 (dt, J=6.0, 1.0 Hz, 1H), 6.99 (t, J=9.0 Hz, 1H), 6.86 (q, J=7.2 Hz, 0.73H), 4.28 (m, 0.26H), 3.66-3.51 (m, 16H), 3.34 (s, 3H), 2.26 (m, 0.47H), 1.24 (d, J=7.3 Hz, 3H). ¹³C NMR (126 MHZ, CDCl₃) δ 162.75, 162.30, 16.14 (d, J=250.8 Hz), 138.87, 134.68, 133.91, 133.49, 133.00, 131.99 (d, J=8.3 Hz), 131.77, 131.40, 131.32, 128.67 (d, J=12.3 Hz), 124.56 (d, J=3.2 Hz), 123.60, 120.72, 116.16 (d, J=21.5 Hz), 72.00, 70.70, 70.68, 70.60, 70.57, 70.47, 70.16, 59.07, 49.98, 38.71, 15.10. ¹⁹F NMR (471 MHz, CDCl₃) δ −112.07 (minor rotamer), −112.33 (major rotamer).

### Example XXIII

GAB_{A}A receptor binding

| Compound | GABA_{A}R binding (IC50) nM |
|---|---|
| PI320 | 242 |
| PI350 | 58 |
| PI531 | 31 |
| PI352 | 38 |

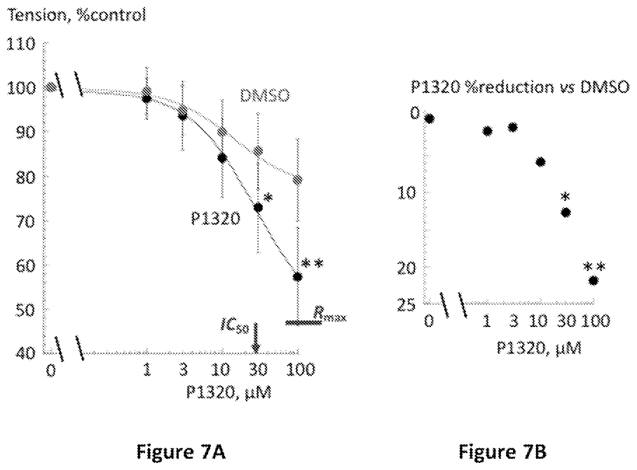

GAB_{A}A receptor binding: Rat brain membranes were prepared from frozen tissue that was thawed on ice and homogenized on ice in 10 volumes of cold lysis buffer (50 mM Tris HCl, pH 7.4, containing protease inhibitor cocktail; Roche) using a Polytron homogenizer (6 pulses and 10 seconds per pulse). The homogenate was centrifuged at 1,000×g for 10 min at 4° C. to obtain the supernatant. The supernatant was then centrifuged at 40,000×g for 20 min, and the resulting supernatant decanted and replaced with the same ice-cold lysis buffer. Two or three additional rounds of homogenization-centrifugation were performed to ensure thorough homogenization and wash out endogenous ligands. The final pellet was resuspended in the same buffer and homogenized one last time. The rat brain suspension was diluted in buffer (50 mM Tris HCl, 2.5 mM CaCl2, pH 7.4), followed by the addition of [3H]-flunitrazepam (0.6-4.0 nM in DMSO) and PI320 or PI310 in DMSO at different concentrations to reach a final of volume of 125 μl per well. Total binding and nonspecific binding were determined with reference compound clonazepam. In brief, plates are usually incubated at room temperature and in the dark for 90 min. Reactions are stopped by vacuum filtration onto 0.3% polyethyleneimine (PEI) soaked 96-well filter mats using a 96-well Filtermate harvester, followed by three washes with cold PBS buffer. Scintillation cocktail was then melted onto the microwave-dried filters on a hot plate and radioactivity counted in a Microbeta counter. The data (n=6) were analyzed by nonlinear regression. See FIG. 7A and FIG. 7B which show a concentration-dependent reduction of force with P1320.

### Example XXIV

### Functional Analysis in Overactive Bladder Model

C57BL/6 female mice (12 weeks, Harlan UK, Ltd) are euthanized by cervical dislocation. The bladder is removed after a midline laparotomy, the neck and trigone region cut away and the dome laid out as a sheet after an anterior wall incision. Strips with intact mucosa (5-7 mm length, 1-2 mm diameter) are dissected and tied in a horizontal trough between a fixed hook and an isometric force transducer. Preparations are superfused (3 ml·min−1) at 36° C. with Tyrode's solution. PI320 or DMSO vehicle are added to the superfusate and their effects on tension measured.

Unstimulated preparations are allowed to equilibrate under tension (20 mN) until a stable base-line is recorded. The preparation is exposed to 10 UM carbachol and peak and steady-state tension recorded. In the continued presence of carbachol the preparation is also exposed to P1320 in cumulatively increasing concentrations (1, 3, 10, 130, 100 uM) until a new steady-state is achieved at each concentration. For vehicle controls DMSO is added in rising concentrations (0.01, 0.03, 0.1, 0.3, 1.0% (v/v)) after a steady-state contraction is achieved with carbachol.

Data are mean±SD; differences between multiple data sets were tested with repeated measures two-way ANOVA and Tukey post hoc tests.

### REFERENCES

1. Kaplan, G. G. (2015) “The Global Burden of Ibd: From 2015 to 2025,” Nat. Rev. Gastroenterol. Hepatol. 12 (12), 720-727.
2. Masiulis, S. et al. (2019) “Gabaa Receptor Signalling Mechanisms Revealed by Structural Pharmacology,” Nature 565 (7740), 454-459.
3. Lim, A. G. et al. (1999) “Diversion Colitis: A Trigger for Ulcerative Colitis in the in-Stream Colon?,” Gut 44 (2), 279.
4. Sinagra, E. et al. (2016) “Inflammation in Irritable Bowel Syndrome: Myth or New Treatment Target?,” World J. Gastroenterol. 22 (7), 2242-2255.
5. Salzmann, J. L. et al. (1989) “Morphometric Study of Colonic Biopsies: A New Method of Estimating Inflammatory Diseases,” Laboratory Investigation 60 (6), 847-851.
6. Cerilli, L. A. and Greenson, J. K. (2012) “The Differential Diagnosis of Colitis in Endoscopic Biopsy Specimens: A Review Article,” Arch. Pathol. Lab. Med. 136 (8), 854-864.
7. Powell, D. W. et al. (2011) “Mesenchymal Cells of the Intestinal Lamina Propria,” Annu. Rev. Physiol. 73 (1), 213-237.
8. Høiby, N. et al. (2010) “Pseudomonas Aeruginosa Biofilms in Cystic Fibrosis,” Future Microbiol. 5 (11), 1663-1674.
9. Busse, W. W. et al. (2010) “Role of Viral Respiratory Infections in Asthma and Asthma Exacerbations,” Lancet (London, England) 376 (9743), 826-834.
10. Bieber, T. (2008) “Atopic Dermatitis,” N. Engl. J. Med. 358 (14), 1483-1494.
11. Drucker, A. M. et al. (2017) “The Burden of Atopic Dermatitis: Summary of a Report for the National Eczema Association,” Journal of Investigative Dermatology 137 (1), 26-30.
12. Nutten, S. (2015) “Atopic Dermatitis: Global Epidemiology and Risk Factors,” Ann. Nutr. Metab. 66 Suppl 1, 8-16.
13. Eichenfield, L. F. et al. (2014) “Guidelines of Care for the Management of Atopic Dermatitis,” J. Am. Acad. Dermatol. 70 (2), 338-351.
14. Hanifin, J. M. et al. (2007) “A Population-Based Survey of Eczema Prevalence in the United States,” Dermatitis® 18 (2).
15. Carroll, C. L. et al. (2005) “The Burden of Atopic Dermatitis: Impact on the Patient, Family, and Society,” Pediatr. Dermatol. 22 (3), 192-199.
16. Silverberg, J. I. et al. (2015) “Sleep Disturbances in Adults with Eczema Are Associated with Impaired Overall Health: A US Population-Based Study,” Journal of Investigative Dermatology 135 (1), 56-66.
17. Halvorsen, J. A. et al. (2014) “Suicidal Ideation, Mental Health Problems, and Social Function in Adolescents with Eczema: A Population-Based Study,” Journal of Investigative Dermatology 134 (7), 1847-1854.
18. Silverberg, N. B. and Silverberg, J. I. (2015) “Inside out or Outside In: Does Atopic Dermatitis Disrupt Barrier Function or Does Disruption of Barrier Function Trigger Atopic Dermatitis?,” Cutis 96 (6), 359-361.
19. Hahn, E. L. and Bacharier, L. B. (2005) “The Atopic March: The Pattern of Allergic Disease Development in Childhood,” Immunol. Allergy Clin. North Am. 25 (2), 231-246.
20. van der Hulst, A. E. et al. (2007) “Risk of Developing Asthma in Young Children with Atopic Eczema: A Systematic Review,” J. Allergy Clin. Immunol. 120 (3), 565-569.
21. Spergel, J. M. (2010) “Epidemiology of Atopic Dermatitis and Atopic March in Children,” Immunol. Allergy Clin. North Am. 30 (3), 269-280.
22. Silverberg, N. B. (2016) “A Practical Overview of Pediatric Atopic Dermatitis, Part 2: Triggers and Grading,” Cutis 97 (5), 326-329.
23. Zahn, N. M. et al. (2020) “Nebulized Midd0301 Reduces Airway Hyperresponsiveness in Moderate and Severe Murine Asthma Models,” ACS Pharmacology & Translational Science 3 (6), 1381-1390.
24. ten Hacken, N. H. et al. (1998) “Elevated Serum Interferon-Gamma in Atopic Asthma Correlates with Increased Airways Responsiveness and Circadian Peak Expiratory Flow Variation,” Eur. Respir. J. 11 (2), 312-316.
25. Chang, P.-J. et al. (2015) “Impaired Nuclear Translocation of the Glucocorticoid Receptor in Corticosteroid-Insensitive Airway Smooth Muscle in Severe Asthma,” American Journal of Respiratory and Critical Care Medicine 191 (1), 54-62.
26. Li, J. J. et al. (2010) “Il-27/Ifn-ΓInduce Myd88-Dependent Steroid-Resistant Airway Hyperresponsiveness by Inhibiting Glucocorticoid Signaling in Macrophages,” The Journal of Immunology 185 (7), 4401-4409.
27. Cozzi, E. et al. (2011) “The Naïve Airway Hyperresponsiveness of the a/J Mouse Is Kit-Mediated,” P.N.A.S. 108 (31), 12787-12792.
28. Naidoo, K. et al. (2018) “Eosinophils Determine Dermal Thickening and Water Loss in an Mc903 Model of Atopic Dermatitis,” Journal of Investigative Dermatology 138 (12), 2606-2616.
29. Li, M. et al. (2006) “Topical Vitamin D3 and Low-Calcemic Analogs Induce Thymic Stromal Lymphopoietin in Mouse Keratinocytes and Trigger an Atopic Dermatitis,” P.N.A.S. 103 (31), 11736-11741.
30. Kim, B. S. et al. (2013) “Tslp Elicits Il-33-Independent Innate Lymphoid Cell Responses to Promote Skin Inflammation,” Sci. Transl. Med. 5 (170), 170ra116-170ra116.
31. Vandeghinste, N. et al. (2018) “Neutralization of Il-17c Reduces Skin Inflammation in Mouse Models of Psoriasis and Atopic Dermatitis,” Journal of Investigative Dermatology 138 (7), 1555-1563.
32. Liu, J. et al. (2008) “A High-Throughput Functional Assay for Characterization of Gamma-Aminobutyric Acid (a) Channel Modulators Using Cryopreserved Transiently Transfected Cells,” Assay Drug Dev Technol 6 (6), 781-786.
33. Im, W. B. et al. (1994) “Effects of GABA and Various Allosteric Ligands on Tbps Binding to Cloned Rat GABA (a) Receptor Subtypes,” Br J Pharmacol 112 (4), 1025-1030.
34. Sur, C. et al. (1998) “Rat and Human Hippocampal Alpha5 Subunit-Containing Gamma-Aminobutyric Acida Receptors Have Alpha5 Beta3 Gamma2 Pharmacological Characteristics,” Mol Pharmacol 54 (5), 928-933.
35. Hauser, C. A. et al. (1997) “Flunitrazepam Has an Inverse Agonistic Effect on Recombinant Alpha6beta2gamma2-Gabaa Receptors Via a Flunitrazepam-Binding Site,” J Biol Chem 272 (18), 11723-11727.
36. Chuang, S. H. and Reddy, D. S. (2018) “Genetic and Molecular Regulation of Extrasynaptic GABA-a Receptors in the Brain: Therapeutic Insights for Epilepsy,” The Journal of pharmacology and experimental therapeutics 364 (2), 180-197.
37. Zahn, N. M. et al. (2019) “Midd0301-a First-in-Class Anti-Inflammatory Asthma Drug Targets Gabaa Receptors without Causing Systemic Immune Suppression,” Basic Clin Pharmacol Toxicol 125 (1), 75-84.
38. Roni, M. S. R. et al. (2020) “The Effects of pH on the Structure and Bioavailability of Imidazobenzodiazepine-3-Carboxylate Midd0301,” Mol. Pharm. 17 (4), 1182-1192.
39. Middendorp, S. J. et al. (2014) “Relative Positioning of Classical Benzodiazepines to the T2-Subunit of Gabaa Receptors,” ACS Chem. Biol. 9 (8), 1846-1853.
40. Pandey, K. P. et al. (2020) “Design, Synthesis and Characterization of Novel Gamma-Aminobutyric Acid Type a Receptor Ligands,” Arkivoc 2020 (Pt 7), 242-256.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in mycology, molecular biology, biochemistry, chemistry, botany, and medicine, or related fields are intended to be within the scope of the following claims.

1. A compound having the structure: 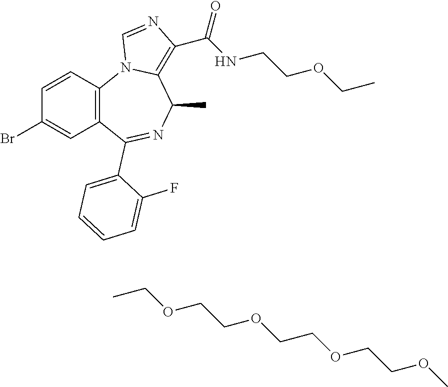
or a pharmaceutically acceptable salt thereof.

2. A method of reducing inflammation comprising administering an effective amount of the compound of claim 1, or a pharmaceutically acceptable salt thereof, to a subject in need thereof.

3. The method of claim 2, wherein said subject has inflammation of the gastrointestinal tract.

4. The method of claim 3, wherein said administering is oral.

5. The method of claim 2, wherein said subject has inflammation due to an autoimmune disease.

6. The method of claim 5, wherein said autoimmune disease is inflammatory bowel disease.

7. The method of claim 6, wherein said inflammatory bowel disease is selected from the group consisting of Crohn's disease and ulcerative colitis.

8. The method of claim 2, wherein said subject has an inflammatory skin disorder.

9. The method of claim 8, wherein said inflammatory skin disorder is atopic dermatitis.

10. The method of claim 2, wherein said subject has an inflammatory lung disease.

11. The method of claim 10, wherein said inflammatory lung disease is asthma.

12. A method of treating overactive bladder syndrome comprising administering an effective amount of the compound of claim 1, or a pharmaceutically acceptable salt thereof, to a subject in need thereof.

13. A compound having the structure: 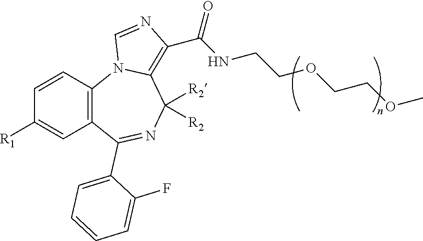
or a pharmaceutically acceptable salt thereof, wherein
wherein:
R₁ is H, Cl, Br, —OCH₃, —C≡CH, or cyclopropyl
R₂ and R₂′ are each independently H, D, C₁₋₄alkyl, cyclopropyl; or R₂ and R₂′, together form a substituted or unsubstituted ring, and
n is any number between 1 and 15.

14. The compound of claim 13, having the structure: 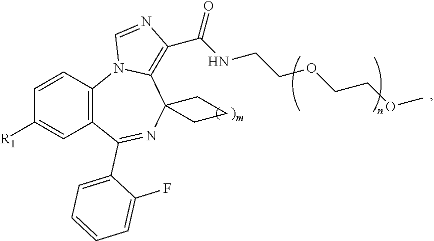
wherein m is any number between 0 and 3.

15. A method of reducing inflammation comprising administering an effective amount of the compound of claim 13, or a pharmaceutically acceptable salt thereof, to a subject in need thereof.

16. The method of claim 15, wherein said subject has inflammation of the gastrointestinal tract.

17. The method of claim 15, wherein said administering is oral.

18. The method of claim 15, wherein said subject has inflammation due to an autoimmune disease.

19. The method of claim 18, wherein said autoimmune disease is inflammatory bowel disease.

20. The method of claim 19, wherein said inflammatory bowel disease is selected from the group consisting of Crohn's disease and ulcerative colitis.

21. The method of claim 15, wherein said subject has an inflammatory skin disorder.

22. The method of claim 21, wherein said inflammatory skin disorder is atopic dermatitis.

23. The method of claim 15, wherein said subject has an inflammatory lung disease.

24. The method of claim 23, wherein said inflammatory lung disease is asthma.

25. A method of treating overactive bladder syndrome comprising administering an effective amount of the compound of claim 13, or a pharmaceutically acceptable salt thereof, to a subject in need thereof.

26. The method of claim 25, wherein said subject is a human.

27. The method of claim 25, wherein said subject is an animal.
